(19) United States
(12) Patent Application Publication
(10) Pub. No.: US 2026/0232299 A1
(43) Pub. Date: Aug. 13, 2026

(54) A SAMPLING APPARATUS, A MEDICAL DEVICE, AND A SAMPLE COLLECTION DEVICE

(71) Applicant: CONCEPTOMED AS, Ballstad (NO)

(72) Inventors: Christian Mide, Ballstad (NO); Jostein Tveide, Ballstad (NO)

(21) Appl. No.: 19/154,648

(22) PCT Filed: Feb. 7, 2023
(86) PCT No.: PCT/EP2024/053100

§ 371 Date: Aug. 7, 2025

(30) Foreign Application Priority Data
Feb. 7, 2023 (GB) 2301727.0

(51) Int. Cl. A61B 10/00

(52) U.S. Cl. CPC A61B 10/0051; A61B 2010/0006

(57) ABSTRACT

A sampling apparatus comprising: a sample collection device (4) comprising a sample collection element (8) for the collection of a sample; and a sample processing device (6) comprising a cavity shaped to receive at least a portion of the sample collection device (8). The cavity comprises a first portion closed off by a first breakable seal (56). The cavity is shaped such that when the sample collection device (4) is inserted into a first position in the cavity, the sample collection device (4) breaks the first breakable seal (56) so as to allow a sample collected by the sample collection element (8) to pass into the first portion of the cavity.

The present invention relates to sampling apparatuses, sample collection devices, kits of parts and medical devices.

In some medical applications, samples are collected and analysed e.g. for diagnostic purposes. In the exemplary case of self-testing for SARS-COV-2 (Covid-19), a relatively complex procedure is carried out involving the collection and analysis of a sample. In some test procedures, a specific volume of a buffer agent is first measured out by a user into a pot. A sample is then collected. The sample may, for example, comprise a nasal sample which is obtained using a nasal swab. Such a nasal sample may be obtained by placing the nasal swab into the sample provider's nasal passage, rotating the nasal swab to obtain the nasal sample, before then removing the nasal swab. The nasal swab may then be placed into the pot containing the buffer, thereby allowing the buffer and the sample to mix. Once the sample has mixed with the buffer, the mixture may be dispensed onto a lateral flow test which may function to analyse the sample.

There are a number of potential problems with the testing procedure described above. When measuring out the buffer agent, it is possible that a user may measure out an incorrect volume of buffer agent, which may impact the test being performed. Additionally, in measuring out and dispensing the buffer agent into the collection pot, there is a risk that the buffer agent may become contaminated, through cross-contamination from the surroundings, thereby providing a degree of uncertainty as to the origin of a viral agent being tested for. Similarly, the pot may have an open top, and thus the buffer may become contaminated in the pot during the time in which the rest of the test is being performed, e.g. whilst a sample is being collected.

Further, when a user dispenses the sample and buffer mixture onto the lateral flow test, there is a risk that they may not dispense enough, or may dispense too much, of the mixture. Dispensing of the incorrect volume of the mixture onto the lateral flow test may impact the ability for the lateral flow test to accurately indicate the presence of a biomarker within the mixture, as the potential variance in buffer volume may result in a different ratio of sample to buffer.

The present invention aims to address or at least mitigate at least one of the problems set out above. When viewed from a first aspect, the present invention provides a sampling apparatus comprising:
a sample collection device comprising a sample collection element for the collection of a sample; and
a sample processing device comprising a cavity shaped to receive at least a portion of the sample collection device, wherein:
   the cavity comprises a first portion closed off by a first breakable seal; and
   wherein the cavity is shaped such that when the sample collection device is inserted into a first position in the cavity, the sample collection device breaks the first breakable seal so as to allow a sample collected by the sample collection element to pass into the first portion of the cavity.

The sampling apparatus may thus be used for the collection and, at least partial, processing of a sample. The presence of the first penetrable seal which effectively acts to partition the cavity provides a convenient means for controlling the flow of a sample through the sample processing device. As will be appreciated, the sample cannot pass into the first portion of the cavity partitioned by the first breakable seal until said seal has been broken. The first portion may be considered to be a volume or space within the sample processing device. The breakable seal may be configured to be torn and/or ruptured. The breakable seal may be penetrable. The breakable seal may be formed from a material which is impermeable to fluid, e.g. liquid and/or air.

In some embodiments, the first portion of the cavity may be open ended. In other words, the first portion of the cavity may be closed at one end by the first breakable seal and be open at another end (e.g. the opposite end). As such the cavity may have a first, open end, a second, open end, separated by the first breakable seal arranged at some point within the cavity between the first and second open ends. In such embodiments, the cavity may be considered to form a conduit extending through the sample processing device. The sample processing device may comprise a housing, and the cavity may be at least partially formed within the housing. In some embodiments, the cavity comprises a proximal end, into which the sample collection element is inserted into the cavity, and a distal end. In some embodiments, the distal end may be closed. The closed distal end together with the first breakable seal may define the first portion of the cavity, which may be a closed (i.e. a fully contained) portion of the cavity.

In some embodiments, wherein the cavity is formed within the housing, the closed distal end may be defined by an integrally formed part of the housing. As such, the housing may comprise an integrally formed wall which defines the closed distal end. In other embodiments, a closure member may be attached to the housing to close the distal end of the cavity. The closure member may take any suitable form. In some embodiments, the closure member may comprise a collection tube. Such a collection tube may be an off-the-shelf component. The collection tube may have a closed end and an open end, wherein the open end is attached to the sample housing. When attached to the housing, the closure member may be considered to form part of the sample processing device. The closure member may comprise a hollow chamber which at least partially defines the cavity of the sample collection device when it is attached to the housing.

In some embodiments, the closure member may be attached to the housing at the distal end. In other embodiments, the closure member may be attached to the housing at a position on the housing spaced along the housing from the distal end. As such, the distal end of the housing may extend at least partially into the closure member. This may advantageously reduce the overall length of the apparatus when the closure member is attached, thus making the apparatus more compact, at least when compared to embodiments wherein the closure member is attached to a distal end of the housing. This may ensure that a typical closure member, e.g. in the form of a collection tube, can be used which may advantageously be used in a wide variety of laboratory settings. The housing may comprise a threaded portion arranged at a point on the housing between the distal end and proximal end of the housing, and the closure member may comprise a corresponding threaded portion arranged to engage with the threaded portion on the housing. The threaded portion may be arranged closer to the proximal end of the housing than the distal end. The corresponding threaded portion may be arranged at a proximal end of the closure member. A tamper element may be arranged between the closure member and the housing. The tamper element may be configured such that it has to be removed and/or broken in order to separate the closure member from the housing. The tamper element may thus provide an indication to the user as to whether the closure member and the housing have been previously separated.

The first breakable seal may function to close off the first portion of the cavity for any appropriate reason. In a set of embodiments, the first breakable seal contains a reagent within the first portion of the cavity. In such embodiments, the first portion of the cavity may be a closed portion which may be closed by an integrally formed part of a housing (which defines the cavity) or by a closure member attached to the housing, as set out above. The presence of a reagent contained within the cavity by the first breakable seal, coupled with the sample collection device which is capable of breaking the first breakable seal, may provide a convenient means for selectively controlling mixing of the sample and a specific volume of reagent. As will be appreciated, a fixed, predetermined volume of reagent may be contained within the sample processing device such that a user does not have to manually measure a volume of reagent for mixing with the sample. The sampling processes may thus be simpler and more accurate, and the apparatus may also minimise the risk of contamination and cross-contamination of the reagent. When the sample and reagent are allowed to mix, i.e. following penetration of the first breakable seal, this may form a sample and reagent mixture. Containing the reagent within the cavity in this manner may reduce the change of the user coming into contact with the reagent. This may be particularly important as some reagents may be harmful to a user, for example because they are intended to break down a cell membrane. An example of such a reagent is Formaline, which may be used when collecting a sample in the form of a biopsy. Formaline is used as a preservation solution, but it is carcinogenic and highly regulated. Avoiding contact with such a reagent may thus be particularly important.

Whilst it may be useful to contain a reagent within the cavity, this is not always necessary as the sample collection device itself may comprise a reagent for mixing with the sample. Thus, in a set of embodiments, the sample processing device comprises a reagent for mixing with the sample. The reagent may be arranged to mix with the sample in a chamber within the sample collection device. In other embodiments, the reagent may be arranged with the sample collection device, and may only be released to mix with the sample when the sample collection device is inserted into the cavity of the sample processing device. In such embodiments, the sample and reagent may mix within the cavity of the sample processing device. Using the sampling apparatus of the embodiments set out above, it may be possible for a user to mix the sample with the reagent in a manner in which exposure of the sample to the external environment is minimised. This may thus reduce the risk of contamination of the sample.

The sampling apparatus may be a medical sampling apparatus for the collection and processing of a medical sample.

The sample collection device and sample processing device may be physically separate components which can be brought into, and out of, contact with one another. The sample collection device may, e.g. initially, be separate from the sample processing device such that no part of the sample collection device is in contact with the sample processing device. The sample collection device may then be inserted into the cavity of the sample processing device. When the sample collection device is separate to the sample processing device, a user may more easily collect a sample. The sample collection device may initially be separate from the sample processing device in order to collect a sample. In other embodiments, the sample collection device may be pre-attached to the sample processing device in a manner in which at least the sample collection element is shielded from an external environment. This may ensure that the sample collection element, at least, remains as aseptic as possible. This arrangement may thus minimise the amount of packaging that is required in order to store the sampling apparatus. In embodiments wherein the sample collection device is pre-attached to the sample processing device, a tamper element may be arranged between the sample collection device and the sample processing device. The tamper element may be configured to provide an indication once the sample collection device has been at least partially (e.g. fully) separated from the sample processing device. The indication may be a visual indication. For example, the tamper element may comprise an member which partially breaks away from one of the sample collection device and/or sample processing device when the sample collection and processing devices are separated (or at least partially separated) from one another. This may ensure that a user does not inadvertently use an apparatus that has been tampered with or simply used already.

Any suitable part, or indeed all of, the sample collection device may be inserted into the cavity of the sample processing device during use. In some embodiments, the cavity is shaped to receive at least the sample collection element of the sample collection device. In some embodiments, part of the sample collection device may be inserted into the cavity, and part of the sample collection device may sit outside of the cavity. For example, the sample collection element may be inserted into the cavity and a portion of a main body, may sit outside of the cavity. In other embodiments, the cavity is shaped to receive the entire sample collection device therein.

Once a sample has been collected using the sample collection device, the sample collection device may then be inserted into the first position in the cavity of the sample processing device ready for processing. As the sample collection device is moved into the first position, any suitable part of the sample collection device may break, e.g. penetrate, the first breakable seal. The sample collection device may, for example, comprise a dedicated seal breaking element which breaks the first breakable seal as the sample collection device moves into the first position. However, in a set of embodiments, the sample collection element is configured to break the first breakable seal. The sample collection element may thus directly break the first breakable seal. The sample collection element may thus contact and break the first breakable seal when the sample collection device is moved into the first position. The sample collection element may, for example, be suitably rigid so as to be capable of penetrating/breaking the first breakable seal. The sample collection element may protrude outwards from a main body of the sample collection device and may thus be optimally positioned for breaking the first breakable seal. Additionally, use of the part of the sample collection device which holds the sample as the means for directly breaking the first breakable seal may help to ensure that the sample is able to quickly pass into the portion of the cavity partitioned by the first breakable seal. In embodiments wherein the first breakable seal contains a reagent, this may ensure the sample suitably mixes with the reagent as the first breakable seal is broken.

The first breakable seal may take any suitable form that is capable of sealing off the first portion of the cavity, e.g. to contain a reagent or to separate a sample analysis means from the rest of the cavity. The breakable seal may, for example, comprise a thin film. As discussed further below, the first breakable seal may be part of a capsule which is inserted into the cavity.

When the first breakable seal is broken, the sample may then be free to pass into the first portion of the cavity and, for example, mix with the reagent contained therein. In some instances, e.g. to ensure that the sample is thoroughly mixed with the reagent, e.g. so as to ensure appropriate processing of the sample, it may be desirable to shake the sample and within the processing device. Thus, in some embodiments, the sample collection device is configured to seal the cavity at least when the sample collection device is in the first position. The sample collection device may seal the cavity at any suitable point along a length of the cavity. Sealing the cavity may prevent the escape of the sample and, where provided, the reagent. Additionally, sealing the sample (and potentially the sample-reagent mixture) within the cavity may allow the apparatus to be shaken, so as to facilitate mixing. It may also allow the sample and reagent mixture to be safely stored within the apparatus, without risk of it being contaminated, or indeed it contaminating anything else. The sealing may be achieved in any suitable manner. In a set of embodiments, the sample collection device may seal against a wall, e.g. an internal wall, of the cavity. This sealing may be achieved through a friction fitting engagement between the sample collection device and the wall of the cavity. This may comprise sealing against an internal and/or external surface of the wall of the cavity. The sample collection device may act to seal the cavity before it reaches the first position. The sample collection device may comprise a sealing element which seals against the sample processing device.

In embodiments comprising a reagent, either in the sample processing device or within the sample collection device, the sampling apparatus may only be for the purpose of collecting and mixing of the sample with the reagent. In such embodiments, once the first breakable seal has been broken and the sample has mixed with the reagent, the sample collection device may be separated from the sample processing device, and the sample and reagent mixture may be suitably dispensed directly from the cavity. For example, the sample and reagent mixture may be poured out of the cavity, or collected using any suitable means, e.g. a pipette or syringe. In some embodiments, for example those set out above whereby a closure member (e.g. a tube e.g. vial) is attached to a housing of the sample processing device, the tube may be separated from the housing, and the sample (optionally mixed with a reagent) may be poured directly out of the closure member. However, in some instances, it may be desirable to perform further processing of the sample within the sample processing device. In such cases, it may be desirable to allow the sample and reagent mixture to flow into a further part of the sample processing device. Accordingly, in a set of embodiments, the sample processing device further comprises a second breakable seal, spaced from the first breakable seal and arranged to define a chamber within the cavity. In embodiments wherein the sample processing device comprises a housing, the second breakable seal, and thus the chamber, may be arranged within the housing. In embodiments comprising a reagent within the cavity, the chamber may contain the reagent. The presence of the second breakable seal may advantageously allow the sample and reagent mixture to be selectively released from the chamber in which it is contained. This may, for example, allow the sample to be released from the sample processing device entirely, or released into a further section of the sample collection device for further processing. The second breakable seal may be selectively broken by any suitable means. For example, the sample processing device itself may comprise means for selectively breaking the second breakable seal. However, in a further set of embodiments, the sample collection device is configured to break the second breakable seal when the sample collection device is in a second position within the cavity.

Accordingly, movement of the sample collection device into the second position may be used to controllably release the sample and reagent mixture from within the cavity without any contact by the user. The first and/or second breakable seal may take any suitable form which suitably holds the reagent within the cavity and which can be broken so as to allow mixing of the sample and the reagent and in the case of the second breakable seal release of the sample and reagent mixture.

In some embodiments, the sample collection device comprises a plunger configured to push a liquid through the sample processing device. The plunger and/or the sample processing device, e.g. the cavity thereof, may be configured such that the plunger only begins operating to force liquid, i.e. plunge, at a particular point in the movement of the sample collection device relative to the sample processing device. In embodiments comprising a first breakable seal and a second breakable seal, the particular point may be a position at which the first breakable seal has been broken but at which the second breakable seal has not yet been broken. In other embodiments, the plunger may only begin operating after the second breakable seal has been broken. The use of a plunger may facilitate the progression of a sample, and indeed any other liquid, through the sample processing device. In some embodiments, as discussed in further detail below, the plunger may begin to act before the first breakable seal is broken. In embodiments comprising an adaptor, as discussed further below, the plunger may be provided on the adaptor.

Once the sample and reagent have been mixed, it may be necessary to further process the mixture. Accordingly, in a set of embodiments, the sample processing device comprises a processing chamber arranged fluidly downstream of the first breakable seal. The processing chamber may facilitate further processing of the sample and reagent mixture. The processing chamber may be the first portion of the cavity defined by the first breakable seal. In embodiments comprising a second breakable seal, a first chamber may be formed between the first and second breakable seals, and the processing chamber may be arranged adjacent the second breakable seal, and thus adjacent the first chamber. The processing chamber may thus be fluidly downstream of the second breakable seal. As such, when the second breakable seal is broken, the sample and reagent mixture may be free to flow into the processing chamber. In embodiments comprising a reagent within the sample processing device, the reagent may be arranged within the first chamber. The processing chamber may have any suitable form. In some embodiments, the chamber may be an open chamber which is open to an external environment in which the sampling apparatus is present. This may, for example, allow a user to easily access the sample and reagent mixture with a further device. For example, the sample and reagent mixture may flow into the open chamber, and a user may then extract the sample and reagent mixture from the open chamber using any suitable device, e.g. a syringe, a pipette, a swab etc. The processing chamber may have any suitable shape and form, as long as it is capable of receiving a portion of sample (and reagent where provided).

However, in other embodiments, it may be desirable to fully contain the sample and reagent mixture within the apparatus. Accordingly, the processing chamber may comprise a closed chamber. Such a closed chamber may not be open to the external environment. Such a closed chamber may prevent contamination of the sample and reagent mixture, as well as preventing contamination of the environment by the sample and reagent mixture.

It may be desirable, in some instances, to analyse the sample within the apparatus. Accordingly, in a set of embodiments, the sample processing device further comprises a sample analysis means. The sample analysis means may be configured to analyse the sample mixed with a reagent, where provided. The sample analysis means may comprise any suitable means for analysing the sample. For example, it may comprise a lateral flow device (test) or an electrochemical sensor. The sample analysis means may, for example, be configured to indicate the presence of at least one particular biomarker within the sample. Other examples include a visual colour change to paper, or any other suitable sensory output, e.g. a change in small which is detectable by a human. In embodiments comprising a lateral flow device, the lateral flow device may be configured to indicate the presence of at least one biomarker in the sample. In embodiments comprising a processing chamber, at least part of the sample analysis means may be provided in the processing chamber. In the exemplary case of a lateral flow device at least an absorption pad (e.g. porous paper) thereof may be arranged in the processing chamber so as to absorb the sample and reagent mixture. The sample analysis means may be arranged fluidly downstream from the first breakable seal. In embodiments where a second breakable seal is provided, the sample analysis means may be arranged fluidly downstream of the second breakable seal.

The Applicant has appreciated that depending on the particular sample being collected and/or the type of analysis which is required, it may not always be possible, or indeed appropriate, to analyse the sample within the apparatus. Instead, it may be necessary to dispense the sample and reagent mixture (where a reagent is provided) onto another device for further processing and/or analysis. Thus, in some embodiments, the sample processing device further comprises a dispensing device for selectively dispensing a liquid, e.g. the sample or a sample and reagent mixture, from the sample processing device. In embodiments comprising a processing chamber, the dispensing device may be provided with, or form part of, the processing chamber. The liquid dispensing device may, for example, comprise a drop counter configured to dispense individual drops of liquid. Such a dispensing device may allow a user to controllably dispense the liquid from the sample processing device, for example, onto an analysis device, e.g. an electronic analysis device or indeed a separate lateral flow test.

It may be desirable to observe the sample within the sample processing device. Thus, in some embodiments, the sample processing device further comprises at least one window for observing the liquid contents of the sample processing device and/or a visual output of an analysis performed within the sample processing device. The at least one window may be provided on the processing chamber. The presence of the at least one window may allow a user to easily observe the analysis of the sample. For example, in embodiments comprising a lateral flow test which comprises a control line and a test line, the at least one window may allow a user to observe the control line and test line. In other embodiments, the at least one window may allow a user to observe the contents of the cavity in which the sample and reagent are mixed. This may allow a user to observe the mixing of the sample and reagent.

In embodiments wherein the sample processing device comprises a reagent, the sample processing device may only comprise a single type of reagent therein. However, in some instances, it may be necessary to mix the sample with different reagents at different points during a sampling procedure so as to suitably process the sample. Accordingly, in a set of embodiments, the cavity comprises at least one further reagent, separated from the reagent. The at least one further reagent may be contained within the cavity by at least one breakable seal which separates the further reagent from the reagent. The at least one breakable seal may similarly be broken by the sample collection device, e.g. the sample collection element as set out above in respect of the first and second breakable seals. As will be appreciated, the type of reagent and/or further reagent may depend on the sample being collected and the type processing of the sample required. In some embodiments, the reagent and/or further reagent comprises a stabilization buffer (e.g. a Universal Transport Medium (UTM)®) configured to stabilize a sample. Other examples of reagents may comprise: nano particles (e.g. magnetic nano-particles) configured to bind to specific targets on nucleic acids, an agent configured to enhance pre-analysis properties of the sample, synthesis primers with desired labels for amplification of nucleic acids, a lysis buffer configured to separate RNA within the fluid sample, and/or any other suitable chemical or biological agent aimed at priming or altering the sample as part of an analysis process. The reagent may be in a liquid or solid form.

The reagent and/or the further reagent may be contained within the cavity in any suitable manner. In some of embodiments, the reagent and/or the further reagent may be contained within a capsule within the cavity. The capsule may comprise and/or define the first and/or second breakable seal or indeed any other breakable seal. As such, the first and/or second breakable seal may be part of the capsule which sits within the cavity of sample processing device. The capsule may thus be considered to be part of the sample processing device. At least a portion of an outer shell of the capsule may form the first, second or indeed any other breakable seal. The use of a capsule which contains the reagent and/or further reagent may allow different reagents to be easily inserted into the cavity of the sample processing device. This may allow different capsules to be inserted depending on the type of processing which is to be performed and/or depending on the type of sample being processed.

It may be desirable to control how far the sample collection device can be inserted into the cavity. Accordingly, in a set of embodiments, at least one of the sample collection device and the sample processing device comprises a stop feature arranged to abut against a part of the other of the sample collection device or the sample processing device when the sample collection device is fully inserted into the sample processing device, so as to prevent further advancement of the sample collection device into the cavity. This may help to prevent a user from over-inserting the sample collection device into the cavity.

The at least part of the sample collection device may be advanced into the cavity of the sample processing device in any suitable manner. For example, the sample collection device may be advanced into the cavity by pushing the sample collection device into the cavity. However, the Applicant has recognised that only pushing the sample collection device may not result in controlled movement of the sample collection device relative to the sample processing device. Thus, in a set of embodiments, the sample processing device comprises a first threaded portion and the sample collection device comprises a second, corresponding threaded portion, each having a first handedness and arranged such that the second threaded portion engages the first threaded portion when the sample collection device is inserted into the cavity and such that rotation of the sample collection device relative to the sample processing device in a first rotational direction advances the sample collection device into the cavity in a first direction. Accordingly, rotation of the sample collection device relative to the sample processing device in a first rotational direction may be used to advance the sample collection device (e.g. the sample collection element thereof) into the cavity of the sample processing device. The first and second threaded portions may be considered to form a first threaded arrangement having a first handedness. Advancing the sample collection device using such a threaded arrangement may allow a user to controllably advance the sample collection device into to the sample processing device.

The first and second threaded portions may be arranged in any suitable manner on each of the sample collection device and sample processing device and may allow the sample collection device to move into any suitable position with respect to the sample processing device. However, in a set of embodiments, the first threaded portion and second corresponding threaded portion are arranged such that rotation of the sample collection device during connection of first threaded portion and second corresponding threaded portion advances the sample collection device into the first position whereby the first breakable seal is broken. As will be appreciated, the sample collection device may thus be controllably inserted into the sample processing device into a position whereby the first breakable seal is broken.

The first and second threaded portions may, for example, be arranged to provide a full range of motion of the sample collection device relative to the sample processing device, e.g. they may be arranged such that rotation during engagement of the first and second threaded portions advances the sample collection device fully into the sample processing device, e.g. into a second position and potentially beyond. However, the Applicant has recognised that it may be advantageous for the first and second threaded portions only to facilitate a limited range of travel of the sample collection device relative to the sample processing device. Thus, in further embodiments, the sample processing device comprises a third threaded portion, and the sample collection device comprises a fourth, corresponding threaded portion, each having a second handedness, different to the first handedness, and arranged such that the fourth threaded portion engages the third threaded portion when the sample collection device is within the cavity and such that rotation of the sample collection device relative to the sample processing device in a second, different, rotational direction advances the sample collection device into the cavity in the first direction.

Accordingly, as will be appreciated by those skilled in the art, despite the third and fourth threaded portions having a different handedness to the first and second portions, when the sample collection device is rotated in the different, i.e. the opposite, rotational direction to the first rotational direction, the sample collection device will nonetheless be advanced in the same first direction into the sample processing device. The third and fourth threaded portions may be considered to be a second threaded arrangement having a second handedness, different to the first handedness. The second and third threaded portions may be arranged such that they engage with one another following engagement of, and rotation through, the first and second threaded portions. Accordingly, a user has to take a conscious step of reversing the direction in which the sample collection device is rotated, following operation between the first and second threaded portions, in order to further advance the sample collection device. This may help to ensure that a user does not advance the sample collection device along its full range of motion inadvertently. The first, second, third and fourth threaded portions may be arranged on their respective devices such that the first and second threaded portions disengage from one another before the third and fourth threaded portions engage with one another.

In a set of embodiments, the third threaded portion and fourth corresponding threaded portion are arranged such that rotation of the sample collection device during connection of third threaded portion and fourth corresponding threaded portion advances the sample collection device into a second position whereby a second breakable seal is broken. As such, the second breakable seal may only be broken once a user has made the conscious decision to reverse the direction of rotation of the sample collection device. This may help to ensure that a user doesn't inadvertently break the second breakable seal.

In another set of embodiments, the third and fourth threaded portions are arranged such that they engage one another following engagement of the first and second threaded portions. As will be appreciated by those skilled in the art, in order to advance the sample processing device into the cavity, the sample processing device may first be inserted into the cavity such that the first threaded portion and second corresponding threaded portion engage with one another. The sample collection device may then be rotated in the first rotational direction such that the sample collection device advances into the cavity in the first linear (e.g. forward) direction into the first position, i.e. at which point the first breakable seal is broken. The first and second threads may then become disengaged, either at the point at which the first breakable seal is broken, or a number of rotations after the seal is broken. The third and fourth threaded portions may then engage with one another and rotation of the sample collection device in the second, different rotational direction, may advance the sample collection device in the same first linear (e.g. forward) direction into the second position whereby the second breakable seal may be broken. This two-stage rotation of the sample collection device in different directions may advantageously allow control of the advancement of the sample collection device and prevent a user from inadvertently breaking both seals without fully mixing the sample with the reagent, where provided. At least the first and second threads may be open ended such that the first and second threads become disengaged from one another once the sample collection device has been rotated by a sufficient amount. The third and fourth threaded portions may be arranged such that they become engaged once the first and second threaded portions become disengaged, e.g. they may be arranged adjacent one another so as to facilitate this subsequent immediate engagement. However, in other embodiments, the third and fourth threaded portions may be suitably arranged on the respective sample processing device and sample collection device in a manner in which once the first and second threaded portions are disengaged, the third and fourth threaded portions are not engaged. Instead, it may be necessary for a user to push the sample collection device by a small amount relative to the sample processing device, so as to advance the sample collection device into a position whereby the third and fourth threaded portions become engaged.

As will be appreciated by those skilled in the art, whilst it is described above how the sample collection device is rotated relative to the sample processing device, this is intended to describe relative rotation of the two components. The sample processing device may instead be rotated relative to the sample collection device. Similarly, both the sample processing device and the sample collection device may be rotated relative to one another.

The threaded portions may be arranged on any suitable part of the respective devices. In a set of embodiments, the first threaded portion is arranged on an inward facing surface of the sample processing device, the third threaded portion is arranged on an outward facing surface of the sample processing device, and wherein the second corresponding threaded portion is arranged on an outward facing surface of the sample collection device and the fourth corresponding threaded portion is arranged on an inward facing surface of the sample collection device. By arranging the respective threaded portions on the inward and outward facing surfaces of the sample collection device and sample processing device, it may be possible to suitably control the interaction between the threads in a manner in which the second threaded portion (provided on the sample collection device) does not engage the third threaded portion (provided on the sample processing device) and similarly such that the fourth threaded portion (provided on the sample collection device) does not engage the first threaded portion (provided on the sample processing device).

The sample collection device may take any suitable form. In a set of embodiments, the sample collection device comprises a main body which comprises a core, with the sample collection element protruding therefrom, surrounded by an outer wall spaced from the core so as to form an annular space around the core. In such embodiments, the second threaded portion may be arranged on an outward facing surface of the core, and the fourth threaded portion may be arranged on an inward facing surface of the outer wall, i.e. a surface facing the core. Similarly, the sample processing device may comprise a tubular outer wall which defines the cavity, and the first threaded portion may be arranged on an inward facing surface of the tubular outer wall, and the third threaded portion may be arranged on an outward facing surface of the tubular outer wall. The outer wall and the core of the sample collection device may be dimensioned such that the core is able to be inserted into the cavity, i.e. into an opening defined by the tubular outer wall of the sample processing device, and such that the annular space defined between the outer wall and the core is suitably dimensioned to receive the tubular outer wall of the sample processing device. As such, the various threaded portions will then be capable of suitably engaging one another during use of the apparatus.

As will be appreciated, the first, second, third and fourth threaded portions may be suitably positioned on the sample collection and sample processing devices in any suitably manner such that their engagement drives the appropriate respective movement. In a set of embodiments, the first and third threaded portions are displaced along an axis of the sample processing device and/or wherein the second and fourth threaded portions are displaced along an axis of the sample analysis device. By displacing the threaded portions in this manner, engagement of the threaded portions may be suitably controlled such that the third and fourth threaded portions are only engaged following engagement of the first and second threaded portions.

In a set of embodiments, the first and second threaded portions have a different pitch to the third and fourth threaded portions. The pitch may determine the amount the sample collection device advances relative to the sample processing device for a given amount of rotation. By having a smaller pitch, more control over the movement of the sample collection device may be achieved.

It may be possible to rotate the sample collection device in at least one reverse direction so as to separate the sample collection device from the sample processing device. However, in some instances, this may not be desirable, for example due to the risk of contaminating the sample, or the risk of contaminating the environment with the sample. Thus, in a set of embodiments, at least one of the sample collection device or the sample processing device comprises a movement retraction means which prevents the sample collection device from being separated from the sample processing device once it has been inserted into the cavity by a predetermined distance. The predetermined distance may correspond to the sample collection device being in the first position. The movement retraction means may thus prevent the sample collection device from being separated from the sample processing device and thus prevent contamination of the sample and reagent mixture and/or prevent contamination of the environment by the sample. The movement retraction means may take any suitable form that prevents separation of the sample collection device from the sample processing device once it has been inserted thereinto. For example, the movement retraction means may comprise a latch on the sample processing device which engages the sample collection device so as to prevents its extraction. In another set of embodiments, at least one of the first threaded portion and second threaded portion comprises the movement retraction means in the form of a ratchet feature which prevents rotation of the first threaded portion and second threaded portion in a direction in which the sample collection device moves out of the cavity. The ratchet feature may be arranged on an end of at least one of the first and second threaded portions such that once the first and second threaded portions become disengaged, they cannot become re-engaged and thus the sample collection device cannot be separated from the sample processing device.

As discussed above, it may be possible to suitably analyse the sample within the sample processing device, e.g. using a lateral flow device. However, depending on the sample or the type of analysis being performed, this may not always be possible. Accordingly, in some embodiments, the apparatus further comprises an electronic analysis device configured to analyse the sample and reagent mixture. The electronic analysis device may comprise a mobile electronic analysis device. The electronic analysis device may, for example, comprise a readout means configured to analyse the sample. The electronic analysis device may allow for enhanced analysis of the sample and reagent mixture. The electronic analysis device may be configured to analyse an output of a lateral flow test.

The sample collection device may be configured to engage directly with the sample processing device when inserted therein. However, in some embodiments, the apparatus comprises an adaptor configured to be attached to a forward end of the sample collection device and to facilitate attachment of the sample collection device to the sample processing device. When attached to the sample collection device, the adaptor may be considered to be part of the sample collection device. Features of the sample collection device discussed previously may thus be applied to the adaptor. The adaptor may thus provide the portion of the sample collection device which is inserted into the cavity of the sample processing device. The adaptor may comprise an engagement means configured to hold the sample collection device and adaptor together. The engagement means may comprise a screw thread arranged on the adaptor configured to engage a screw thread arranged on the sample collection device.

In some embodiments, the adaptor may comprise a locking means configured to prevent the sample collection device from rotating relative to the adaptor, e.g. when the adaptor and sample collection device are fully attached together. In embodiments wherein the sample collection device is rotated relative to the sample processing device in order to advance the sample collection device relative to the sample processing device, the locking means may ensure that rotation of the sample collection device results in rotation of the adaptor, rather than rotation relative to the adaptor.

As described above, in some embodiments, the sample collection device comprises a second threaded portion and optionally a fourth threaded portion. In embodiments comprising an adaptor, the second and optionally the fourth threaded portions may be arranged on the adaptor. The adaptor may thus function to allow sample collection devices which do not have such screw threads to suitably control movement relative to the sample processing device so as to function appropriately with the sample processing device.

In some embodiments, the adaptor may comprise a seal breaking element configured to break at least the first breakable seal. In further embodiments, the seal breaking element may be configured to break any and all breakable seals within the sample processing device. The adaptor may comprise a hollow core, through which a sample may pass through.

In some embodiments, the sample collection element, of the sample collection device, comprises a sample collection conduit. Various optional features of a sample collection device comprising such a sample collection conduit are set out below. The sample collection device may comprise a sample collection chamber, into which the sample collection conduit extends. The sample collection conduit may be movable within the sample collection device. The sample collection conduit may be configured to move between a first position and a second position. In at least the first position, the sample collection conduit may extend to an exterior of the sample collection device. As such, an inlet of the sample collection conduit may be positioned at an exterior of the sample collection device and thus be exposed such that a user can access the inlet, e.g. by placing their mouth around the inlet, so as to provide a sample. The sample collection conduit may thus be suitable for the collection of a saliva sample directly from a user's mouth. The sample collection device may comprise a plunger arranged to expel a sample from within the sample collection chamber. The plunger may be operatively coupled to the sample collection conduit such that movement of the sample collection conduit drives movement of the plunger. The sample collection conduit may be arranged within the sample processing device and be configured to break the first breakable seal. In some embodiments, the sample collection device may comprise an outlet through which a sample may be expelled from the sample collection device. The sample collection conduit may be configured (e.g. by comprising a pointed tip) to break at least one seal within the sample collection device. The seal may, for example, seal an outlet of the sample collection device and/or seal a reagent within the sample collection device. In embodiments comprising a sample collection conduit, a cap may be arranged to drive movement of the conduit. The cap and conduit together may be considered to form the sample collection device. The cap may be act to seal a proximal end of the sample processing device. The sample processing device and cap may be configured to hold the cap in at least one, e.g. a plurality of different, positions relative to the sample processing device.

In some embodiments, the sample collection device may be configured to couple to the sample processing device in a manner in which the at least in one position of the sample collection device relative to the sample processing device, the sample collection device is rotationally coupled to the sample processing device such that rotation of the sample collection device drives rotation of the sample processing device. The position at which this coupling is achieved may be a position in which the sample collection device has been fully received by the sample processing device. This rotation may be in a direction opposite to the direction in which the sample collection device is coupled the sample processing device. This may be achieved by a suitable ratchet arrangement acting between the sample processing device and the sample collection device.

The sampling apparatus may be used to collect and process any suitable sample. In some embodiments, the sampling apparatus is for collection and processing of a biological sample. In some embodiments, the sampling apparatus is configured to collect and process at least one of: blood, saliva, mucosa and body tissue. The sample collection element may be a generic sample collection element that is capable of collecting a number of different forms of sample. In other embodiments, the sample collection element comprises at least one of: a sample collection tube (i.e. a conduit), a capillary tube, a saliva collection tube, a sample collection patch, a sample collection swab, a curette, a syringe and a pipette. As such, the sample collection device may be used to collect a particular sample. For example, the sample collection device may be used to collect a mucosal biopsy, a nasal sample, for the collection of saliva (e.g. using a saliva collection patch or tube), for collecting gingival mucosa, for collecting urine (e.g. using a patch), for blood collection (e.g. using a capillary tube) etc.

The sample collection device may comprise a fixed sample collection element, i.e. such that the sample collection device is suitable for collecting a particular type of sample. In this case, multiple different sample collection devices may be manufactured for the collection of different types of sample. The Applicant has recognised that it may be advantageous to minimise the number of entire sample collection devices which need to be manufactured in order to be able to collect different samples. Accordingly, in some embodiments, the sample collection device comprises a main body (which may be shaped to be held by a user) and wherein the main body comprises a sample collection element receiving portion shaped so as to receive one of a plurality of different sample collection elements. Advantageously, in such embodiments, a single main body can be manufactured that is capable of housing a number of different sample collection elements. This may thus reduce the number of entire sample collection devices which have to be manufactured.

The Applicant has recognised that a sample collection device which comprises such a sample collection element receiving portion shaped to receive one of a plurality of different sample collection elements is novel and inventive in its own right. Thus, when viewed from a further aspect, the present invention provides a sample collection device, for collecting a sample, comprising:
a main body (which may be shaped to be held by a user), wherein the main body comprises a sample collection element receiving portion shaped so as to receive one (at a time) of a plurality of different sample collection elements.

The main body may be shaped to be held by a user's hand, or may be shaped to be held by a piece of equipment, e.g. tongs, or a piece of machinery. The sample collection device, specifically the sample collection element thereof, may be for the collection of a biological sample.

The features set out below may be applied to embodiments of either aspect of the invention set out above. In a set of embodiments, the plurality of different sample collection elements comprise at least two of: a sample collection tube (e.g. a conduit), a capillary tube, a saliva collection tube, a sample collection patch, a sample collection swab, a curette, a syringe and a pipette.

The sample collection element receiving portion may be suitably shaped so as to be capable of securing a sample collection element therein. The sample collection element receiving portion may be shaped to form a friction fit with a sample collection element received therein. The sample collection element receiving portion may comprise a cavity capable of receiving sample collection elements having different dimensions. In other embodiments, the sample collection element receiving portion comprises a receiving cavity which comprises a plurality of sections, each having a different internal dimension (e.g. diameter) so as to be capable of securing different sample collection elements therein. The plurality of sections may, for example, start off having a larger internal dimension at an open (i.e. insertion) end of the sample collection element receiving portion, before gradually reducing in internal dimension towards a distal end of the sample collection element receiving portion.

The sample collection element receiving portion and the sample collection element may together be configured such that irrespective of which sample collection element is inserted into the sample collection element receiving portion, a sample collection portion of the sample collection element extends a fixed distance from an open end of the sample collection element receiving portion. Having the sample collection element protrude from the open end by a fixed distance may advantageously mean that the sample collection device can be used with a single sample processing device, i.e. such that a distinct sample processing device does not have to be manufactured for each type of sample collection device.

As described above, it may be desirable for the sample collection portion of the sample collection element to protrude from an open end of the sample collection element receiving portion by a pre-set amount. However, in some instances, it may also be desirable for the sample collection portion of the sample collection element to protrude from the opening by a larger amount, e.g. to facilitate the collection of a sample. Accordingly, in some embodiments, the sample collection element receiving portion is dimensioned to receive the sample collection element in a first position therein, such that the sample collection element protrudes by a first distance from an end of the sample collection element receiving portion, and further a second position wherein the sample collection element protrudes by a second, reduced distance, from the end of the sample collection element receiving portion. The sample collection element may be pushed between the first and second positions.

The end of the sample collection element receiving portion may be defined by the opening of the sample collection element receiving portion into which the sample collection element is inserted. The sample collection element may comprise a sample collection portion and a support portion which support the sample collection portion. The support portion may be shaped to be inserted into the sample collection element receiving portion and the support portion may be capable of moving within the sample collection element receiving portion so as to allow the sample collection element to move between the first and second positions. The support portion may be rod-shaped. The sample collection element receiving portion may comprise an elongate receiving space capable of receiving the sample collection element. The sample collection element may be moved from the first position to the second position as the sample collection device is inserted into the sample processing device. Any suitable part of the sample processing device may contact the sample collection element and push it into the second position. However, in some embodiments, the first breakable seal is configured to push the sample collection element into the second position, prior to being broken by the sample collection element when the sample processing device reaches the first position.

In further embodiments, the sample collection element receiving portion is configured such that irrespective of the sample collection element which is received therein, when in the second position, the sample collection elements protrudes from the end of the sample collection element receiving portion by the second distance. As discussed above, protruding by the same fixed amount thus ensures that the sample collection device, e.g. the sample collection element thereof, will reliably penetrate the first breakable seal on a single type of sample processing device. As such, the number of different sample processing devices that need to be manufactured may be reduced.

The sample collection element receiving portion may be integrally formed within the main body of the sample collection device. However, in some embodiments, the sample collection element receiving portion is formed in a holder which is received within the main body of the sample collection device, e.g. in a core thereof. Such a holder may provide more design freedom in the design of the main body and may also reduce manufacturing costs.

In other embodiments, the sample collection element may comprise a sample collection portion arranged on the end of a telescoping support arm. As such, the telescoping support arm may be reduced in length such that the sample collection portion of the sample collection element protrudes by the desired amount from the end of the sample collection element receiving portion on the main body.

In some embodiments, the sample collection device comprises a sample collection element received in the sample collection element receiving portion.

In embodiments wherein the sample collection element is arranged to penetrate the first breakable seal, an end of the sample collection element which contacts the first breakable seal may be pointed. For example, the end of the sample collection element may be sharpened. Having a pointed end which contacts the first breakable seal, and optionally the second breakable seal (where included), may reduce the force required to break the breakable seal and thus make operation of the apparatus easier for a user.

In some embodiments, an end of the sample collection element, e.g. a sample collection portion thereof, is exposed so as to be capable of contacting and collecting a sample. The end of the sample collection element may protrude from the rest (e.g. a main body) of the sample collection device. The end of the sample collection element which is exposed may, for example, comprise an open end of a capillary tube, the patch of a sample collection patch, or the scoop/hook of a curette.

In embodiments comprising a capillary tube, the size of the capillary tube may depend on the particular type of sample the sample collective device is designed for collecting. The capillary tube may be dimensioned to collect a liquid sample using capillary action. In some embodiments, the capillary tube may have an internal volume (i.e. a volume of sample which it is capable of collecting) of between 10-100 μl. The internal diameter of the capillary tube may be between 0.50 mm and 1.50 mm, and the outer diameter of the capillary tube may be between 1.25 mm and 1.75 mm. Such capillary tubes may be particularly well suited to the collection of a blood sample. However, as will be appreciated, the dimensions of the capillary tube will depend on the viscosity of the sample being collected.

The sample collection device may be configured to collect a substantially solid sample, e.g. a stool sample. In a set of embodiments, the sample collection element comprises a tip and a surrounding wall which extends around the tip and which defines a sample collection volume between the wall and the tip. The surrounding wall may be an annular wall. The tip and surrounding wall may be define a sample collection portion and be attached to a support portion. The tip and surrounding wall may be arranged at a distal end of the sample collection device. In use, the sample collection element may be pressed into a solid sample, e.g. a stool sample, and the sample may be retained in the sample collection volume between the surrounding wall and the tip. The tip may be pointed and/or sharpened. In embodiments comprising a sample processing device with a breakable seal, the tip may break the breakable seal. The distal end of the sample collection device, i.e. the tip and surrounding wall may be inserted into the sample processing device. The surrounding wall may not, necessarily, be continuous, and there may be breaks within the wall.

In embodiments comprising a sample processing device, the sample processing device may comprise a projecting wall which extends away from the first breakable seal, and the projecting wall and the surrounding wall may be dimensioned such that as the sample collection device is inserted into the cavity of the sample processing device, the tip passes through an inner side of the projecting wall and the surrounding wall passes around an outer side of the projecting wall. The projecting wall and surrounding wall may be dimensioned such that they come into contact with one another. The projecting wall may thus function to separate a sample, e.g. a stool sample, from the inner surface of the surrounding wall. This may ensure that the sample can be easily separated from the sample collection element and thus be suitably processed within the sample processing device.

The sample collection element may be configured to collect a liquid sample. In some embodiments, the sample collection element comprises a substantially rigid tip surrounded by an absorbent body (e.g. an absorbent material), wherein the absorbent body is compressible such that in a first, non-compressed configuration, the tip does not protrude further than an end of the absorbent body and in a second, compressed configuration, the tip protrudes at least as far as (e.g. further than) an end of the absorbent body. When the absorbent body is in the first configuration, no part of the tip may be exposed at the distal end of the sample collection element, the absorbent body may be considered to cover the tip. Such a sample collection element may be configured such that when used with a sample processing device, of any previous embodiment, the absorbent body may initially be in the first, non-compressed configuration, and be caused to become compressed into the second configuration when the sample collection element is inserted into the cavity. When in the second configuration, the tip will be positioned relative to the absorbent body allowing the tip to contact and break the breakable seal. The tip may thus function as a seal breaking element. As with previous embodiments, the tip may be pointed and/or sharpened. The absorbent body may comprise any suitable material, e.g. a sponge.

In further embodiments comprising an absorbent body, the sample collection element may comprise a plunger configured seal against the cavity of the sample processing device. The plunger may be arranged so as to apply a force to the absorbent body, thereby compressing the absorbent body, e.g. when the sample collection element is inserted into the cavity of the sample processing device. The plunger may also act to define the amount of liquid which is dispensed from the absorbent body. This may be defined by the amount of absorbent material which is downstream of the plunger, i.e. between the plunger and a distal end of the sample collection element which is inserted into the sample processing device. The plunger may function to push liquid out of the absorbent body and force the liquid through the sample processing device.

In some instances, in order to obtain a sample, e.g. a blood sample from a person, it may be necessary to puncture a surface, e.g. a person's skin. In some embodiments, the sample collection device comprises a puncture element configured to puncture a surface in order to obtain a sample. In some further embodiments, the puncture element is shielded by a cover, and the puncture element is arranged to move between a retracted position, in which it is it does not protrude from the cover, and a protruding position, in which it protrudes from the cover so as to be capable of puncturing a surface. The puncture element may be configured to move from the retracted position, to the protruding position, and back to the retracted position. This movement may be driven automatically by a movement arrangement. This movement may be driven by a resiliently biased member (e.g. a spring). The puncture element may be caused to move from the retracted position towards the protruding position by the operation of a button. The puncture element may break, puncture, cut or otherwise break into the surface, e.g. a user's skin. The puncture element may comprise a needle or blade. This arrangement set out above may be known as a safety lancet. The puncture element may be in the form of a safety lancet.

The sample collection device may comprise a main body, e.g. in the form of a grip portion or handle, to which the puncture element and the retractable cover may be attached. The puncture element and retractable cover may form part of a sub-assembly which is attached to the main body.

As mentioned previously, any suitable part of the sample collection device may break the breakable seal. In some embodiments, the sample collection device comprises a seal breaking element. The seal breaking element may be a separate component to the sample collection element, and be specifically designed to break the breakable seal, rather than the sample collection element. The Applicant has appreciated that through the provision of a dedicated seal breaking element, more controlled breaking of the breakable seal may be achieved. In some embodiments, the seal breaking element may be configured to break the first breakable seal in a manner in which at least part of the seal remains attached to a wall of the cavity. This may prevent any undesired material (i.e. the material of the seal), from mixing with the sample. The same may be applied to any other breakable seals within the sample processing device.

It may, nonetheless, be desirable for the sample collection element to protrude further from a distal end of the sample collection device than the seal breaking element, at least initially, in order to facilitate the collection of a sample. As such, in some embodiments, the sample collection element is arranged to move relative to the seal breaking element between a collection position in which the sample collection element protrudes further than the seal breaking element, at a distal end of the sample collection device, and a retracted position, in which the seal breaking element protrudes at least as far (e.g. further than) the sample collection element, at the distal end of the sample collection device. It will be appreciated that in this relative movement, the seal breaking element may be fixed, and the sample collection element may move relative to the seal breaking element, or the sample collection element may be fixed, and the seal breaking element may be moved relative to the sample collection element. In some embodiments, both the seal breaking element and sample collection element may be capable of movement. The sample collection device may comprise a handle/grip portion which may define the proximal end of the sample collection device. As an example, the sample collection device may comprise a substantially tubular member an end of which defines the seal breaking element, and the sample collection element may be arranged within a hollow portion of the substantially tubular member and arranged to move within the tubular member between the collection and retracted positions. The hollow portion of the tubular member may be form a sample collection element receiving portion of the sample collection device.

In some embodiments, the sample collection element may comprise a capillary tube. Such a capillary tube may not be sufficiently strong to break the seal, and thus its retraction into the retracted position, thereby putting the sample breaking element at the distal most end of the device, may ensure that the breakable seal can be suitably broken and also minimise the risk of damage to the capillary tube (which may be relatively fragile) occurring. The capillary tube may have any suitable internal volume for collecting a sample, e.g. 50 μl or 20 μl.

The seal breaking element may have any suitable form that is capable of breaking the breakable seal. In some embodiments, the seal breaking element comprises at least one protrusion, e.g. a plurality of protrusions, extending to contact the seal. The protrusions may be in the form of teeth.

The seal breaking element may be in the form of an annular wall, shaped to receive the sample collection element therein. The seal breaking element may thus at least partially define a sample collection element receiving portion.

As discussed previously, in some embodiments, the sample collection element may comprise a curette or swab. Such a curette or swab may be configured to move relative to the seal breaking element as set out above.

The sample collection element may be capable of collecting a viscous material, e.g. mucus. In such embodiments, the sample collection element may comprise a body (i.e. a sample collection portion) which comprises a plurality of at least one of: grooves, slots, holes, protrusions formed therein, configured to increase the surface tension between the body and the sample, and thereby hold the sample against the body. As will be appreciated, a user may simply scrape the body against a sample, and the sample may be retained against the body. This may simplify the collection of some types of sample.

The sample collection device may be configured to collect a biopsy. Accordingly, in some embodiments, the sample collection element comprises a sharpened portion configure to penetrate a surface (e.g. skin) and a sample receiving portion into which a sample is received. The sharpened portion may extend directly into the sample receiving portion. The sample collection element may further comprise an ejection mechanism configured to eject the sample out of the sample receiving portion. This may advantageously allow the sample to be ejected within the sample processing device. The size of the sample receiving portion may depend on the type of biopsy that the device is intended for use with. As an example, the sample receiving portion may have a volume of 2 ml. The ejection mechanism may comprise an ejection button, operable by a user to eject the sample from the sample receiving portion, and wherein the ejection button is arranged on a part of the sample collection device which does not extend into the sample processing device, in use. The ejection button may be operably connected to an ejection rod which may push a sample out of the sample receiving portion. Similarly to the embodiments set out above, the sample collection element and sharpened portion may be configured to move relative to a seal breaking element such that the seal breaking element can become the distal-most part of the device for breaking the breakable seal.

In a set of embodiments, the sample collection element comprises a gripping arrangement comprising first member and second member, wherein at least the first member is configured to move between an open position, in which the first member is spaced from the second member so as to receive, in use, a sample therebetween, and a closed position in which the first member is moved towards the second member so as to grip the sample between the first and second members. Accordingly, the gripping arrangement may be used to select and grip a sample, which may then be further processed. In some embodiments, the second member may also move towards the first member, so as to grip the sample between the first and second members. In the closed position, the first member may contact the second member, or the first member may remain separated from the second member, by a smaller amount than when in the open position. It will be appreciated that the gripping arrangement may comprise further members which are configured to move between the open and closed positions.

In a further set of embodiments, the sample collection device comprises a driving arrangement configured to drive the first member from the open position to the closed position. The driving arrangement may comprise any suitable structure for driving movement of the first member relative to the second member. In a set of embodiments, the driving arrangement comprises a driving structure arranged to drive the first member into the second position. The driving structure may be movably mounted so as to be capable of moving relative to the gripping arrangement, and arranged such that when the driving structure is moved in a first direction (e.g. towards the gripping arrangement), the first member is driven from the open position to the closed position. In some embodiments, the driving structure may be configured to move the first member from the closed position towards (e.g. to) the open position when the driving structure moves in a second direction (e.g. away from the gripping arrangement). In some embodiments, the first member may be resiliently biased into the open position. In such embodiments, when the driving structure is moved in a second direction (e.g. away from the gripping arrangement), the driving structure may permit the first member to move into the open position, under the resilient bias. The gripping arrangement and associated driving structure may thus be used to selectively grip and release a sample. This may, for example, allow a user to grip a sample, and release said sample once the sample collection device is within the sample processing device. It will be appreciated that the relative movement between the driving structure and the gripping arrangement may be achieved by holding the gripping arrangement fixed, and moving the driving structure, holding the driving structure fixed and moving the gripping arrangement, or moving both the driving structure and gripping arrangement relative to one another.

In a set of embodiments, the driving structure is configured to move relative to the gripping arrangement into a position whereby a forward end of the driving structure is at least as far forward as (e.g. further forward than) the gripping arrangement, and wherein the forward end defines a seal breaking element. In such embodiments, the driving structure, specifically the seal breaking element thereof, may thus be used to break the breakable seal of the sample processing device. The seal breaking element may have any of the features of the seal breaking element of any of the embodiments discussed above.

In some embodiments, the driving structure may comprise a tubular portion having a first end and a second end, the device may further comprise a connecting member extending from the gripping arrangement, through the first end and out the second end of the tubular portion, to a gripping member. The driving structure may be configured to move, e.g. slide, relative to the connecting member which extends therethrough. In use, a user may grip the gripping member in one hand, grip the driving structure in another hand, and slide the driving structure relative to the connecting member and thus the gripping arrangement. As the driving structure contacts, or progress along the gripping arrangement (if already in contact), the driving structure moves the first member from the open position towards the closed position. The tubular portion may be configured to receive the gripping portion entirely therein. The tubular portion may thus define the forward end of the driving structure as discussed above. Containing the gripping portion, and thus the sample, entirely within the tubular portion may act to safely temporarily store the sample. It may also minimise the risk of accidental release of the sample, as the driving structure may need to be manually (and actively) moved in order to allow the first member to move into the open position.

In some embodiments, the first member may be pivotally mounted to move relative to the second member. Such an arrangement may be considered to provide a tweezing type of action. The first member may be mounted to move relative to the second member by a living hinge. In some embodiments, the first member may be resiliently biased to move into the open position. This may simplify the format of the driving arrangement, as the driving arrangement only needs to be capable of driving movement of the first member in one direction. The resilient biased may be provided by the living hinge, or a separate resilient bias.

In any of the embodiments described above relating to the gripping member, the second member may also be driven to move relative to the first member. Accordingly, the driving arrangement, and indeed any features thereof, may operate on the second member in a similar manner to that described above in response of the first member.

In some embodiments, the sample processing device comprises a filter arranged within the cavity to filter the sample. The filter may comprise a fine mesh. The size of the mesh, i.e. the size of material which it prevents from passing through, may depend on the particular application of the apparatus. The filter may be arranged fluidly downstream of the breakable seal.

The Applicant has appreciated that a number of the features of the sample collection devices set out above are novel and inventive in their own right. Therefore, according to a further aspect of the present invention there is provided a sample collection device comprising a sample collection element, the sample collection device (e.g. the sample collection element) having any of the features of the various sample collection devices set out above.

According to a further aspect of the present invention there is provided, a kit of parts comprising:
a sample collection device of any of the embodiments discussed above configured to be capable of receiving a plurality of different sample collection elements; and
a plurality of different sample collection elements.

The kit of parts may thus facilitate the collection of a number of different samples. The plurality of sample collection elements may comprise at least two of: a sample collection tube, a capillary tube, a saliva collection tube, a sample collection patch, a sample collection swab, a curette, a syringe, a pipette and a gripping arrangement. In some embodiments, the kit of parts further comprises
a sample processing device. The sample processing device may comprise any of the features of the sample processing device in accordance with the aspects of the invention above. The kit of parts may further comprise a plurality of capsules each comprising different reagents and/or different volumes of reagents. The plurality of capsules comprising different reagents and/or different volumes of reagents may allow a user to select a particular reagent that is required for a particular form of processing and/or depending on the sample that has been obtained. The kit of parts may further comprise a sample processing device having any of the features of the embodiments set out above.

Further, in relation to use of the threaded portions discussed above, the Applicant has recognised that advancing a first body, i.e. the sample collection device, relative to a second body, i.e. the sample processing device, by rotation of the first body around a first rotational direction and then rotation in a second rotational direction to achieve advancement in a single linear direction is novel and inventive in its own right. Accordingly, when viewed from a second aspect, the present invention provides a medical device comprising:
a first body and a second body, wherein the first body is arranged to move relative to the second body;
a first thread arrangement configured between the first body and the second body such that rotation of the first body in a first rotational direction relative to the second body advances the first body in a first linear direction; and
a second thread arrangement configured between the first body and the second body such that rotation of the first body in a second direction opposite to the first rotational direction advances the first body in the same first linear direction.

As such, the first body may be moved relative to the second body in the same first linear direction (e.g. a forward direction) by both rotation in a first rotational direction and rotation in a second different direction. As such, following rotation in a first direction, in order to continue advancing the first body in the same direction, the user has to change the direction of rotation of the first body. The first thread arrangement and second thread arrangement may be configured such that only the first thread arrangement or second thread arrangement is engaged at any one time. The first body and second body may comprise any two components of a medical device where selective control over their relative movement is required.

The first and second thread arrangements may have any suitable form. In a set of embodiments, the first thread arrangement comprises a first threaded portion arranged on the first body and a second corresponding threaded portion on the second body;
the second thread arrangement comprises a third threaded portion arranged on the first body and a fourth threaded portion arranged on the second body; and wherein
the first and second threaded portions have an opposite handedness to the third and fourth threaded portions.

In a set of embodiments, the first and second threaded portions have a different pitch to the second and third threaded portions. The pitch may determine the amount of linear movement for a given rotational movement of the bodies. In some embodiments, the second thread arrangement is configured such that it is engaged following engagement, and subsequent disengagement, of the first thread arrangement. In other words, the first body is moved relative to the second body first by engagement of the first thread arrangement, at which point the first thread arrangement becomes disengaged, and the second thread arrangement can become engaged.

In a further set of embodiments, the first and third threaded portions are displaced along on the first body in a direction aligned with the first linear direction and/or wherein the second and fourth threaded portions are displaced along the second body in a direction aligned with the first linear direction. Displacing the first and third threaded portions and/or the second and fourth threaded portions may control the point during linear movement of the first body relative to the second body at which the first and second threaded portions engage with one another and the point at which the third and fourth threaded portions engage with one another.

In a set of embodiments, one of the first and third threaded portions is arranged on an inward facing surface of the first body and the other of the first and third threaded portion is arranged on an outward facing surface of the first body, and one of the second and fourth threaded portions is arranged on an inward facing surface of the second body and the other of the second and fourth threaded portions is arranged on an outward facing surface of the second body.

In some embodiments, the first and second bodies are separable from one another. The first and second bodies may initially be separate from one another, prior to engagement of the first thread arrangement. In other embodiments, the first and second bodies are attached to one another in a permanent manner such that they cannot be separated from one another.

In some embodiments, the first body comprises a sample collection device and the second body comprises a sample processing device. As such, the first and second bodies may comprise any of the features set out above in respect of the earlier aspects of the present invention.

Whilst it is described above how the first body is rotated relative to the second body, it will be appreciated that this refers to relative rotation and the second body may instead be rotated relative to the first body, or both of the first and second bodies may be rotated with respect to one another.

In any the embodiments described above comprising a first breakable seal, said seal may be replaced with a sealing element arranged to seal a respective portion of the cavity and wherein the sealing element is capable of adopting an open configuration. In such embodiments, instead of breaking the seal, the sample collection device may be configured to move the seal element into the open configuration. The same applies to the second breakable seal, or indeed any other breakable seal within the apparatus.

Some preferred embodiments of the present invention will now be described, by way of example only, and with reference to the following drawings, in which:

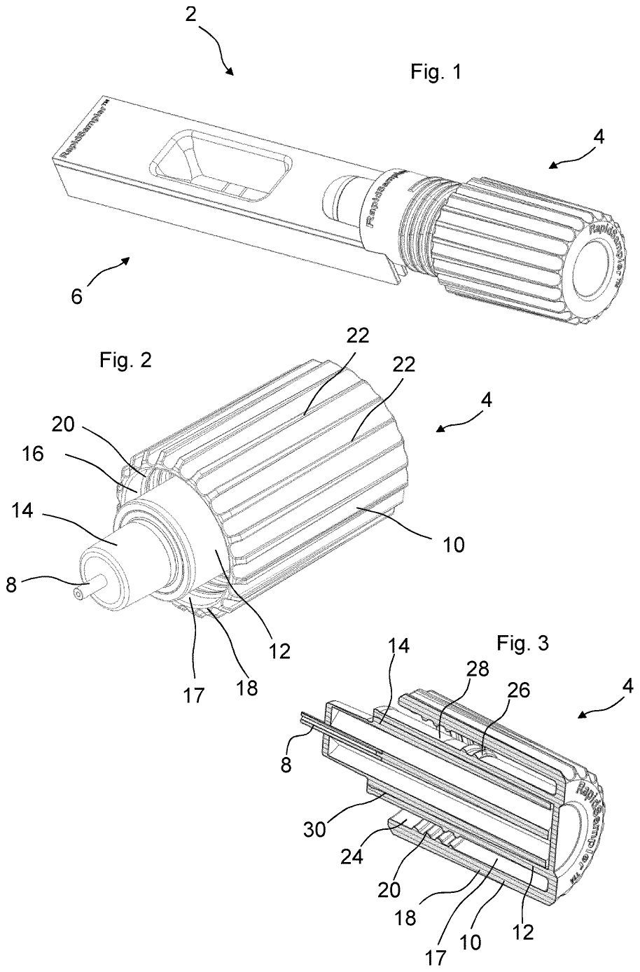

FIG. 1 shows a perspective view of a sampling apparatus in accordance with an embodiment of the present invention;

FIG. 2 shows a perspective view of the sample collection device of the apparatus shown in FIG. 1;

FIG. 3 shows a cross-sectional view through the sample collection device shown in FIG. 2;

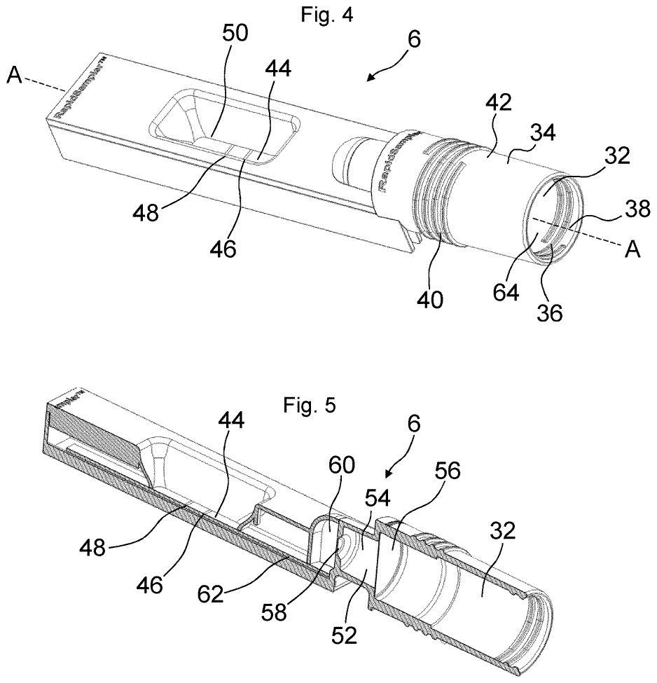

FIG. 4 shows a perspective view of the sample processing device of the apparatus shown in FIG. 1;

FIG. 5 shows a cross-sectional view through the sample processing device shown in FIG. 1;

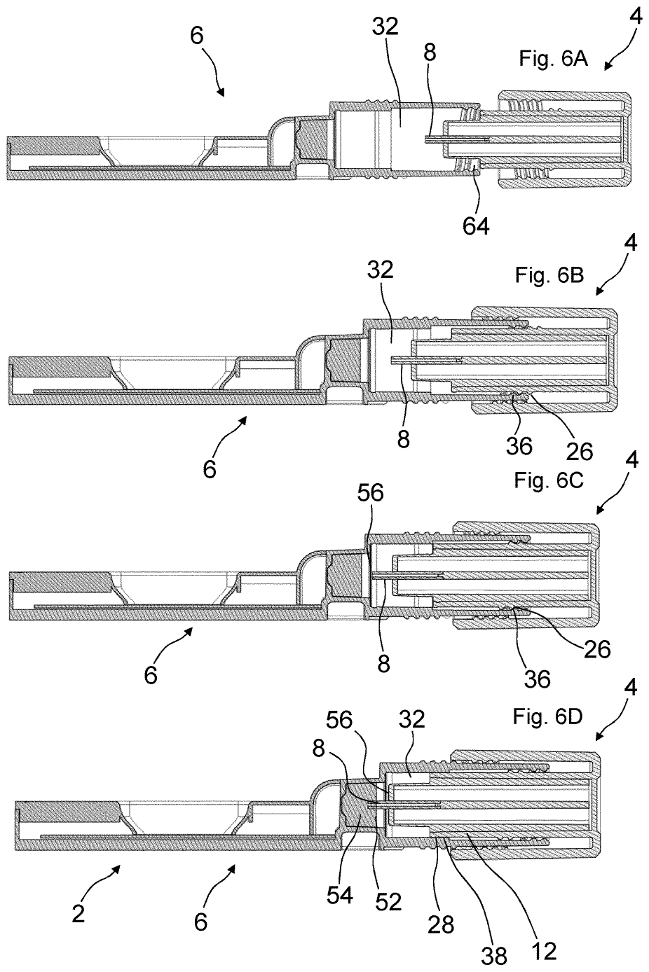

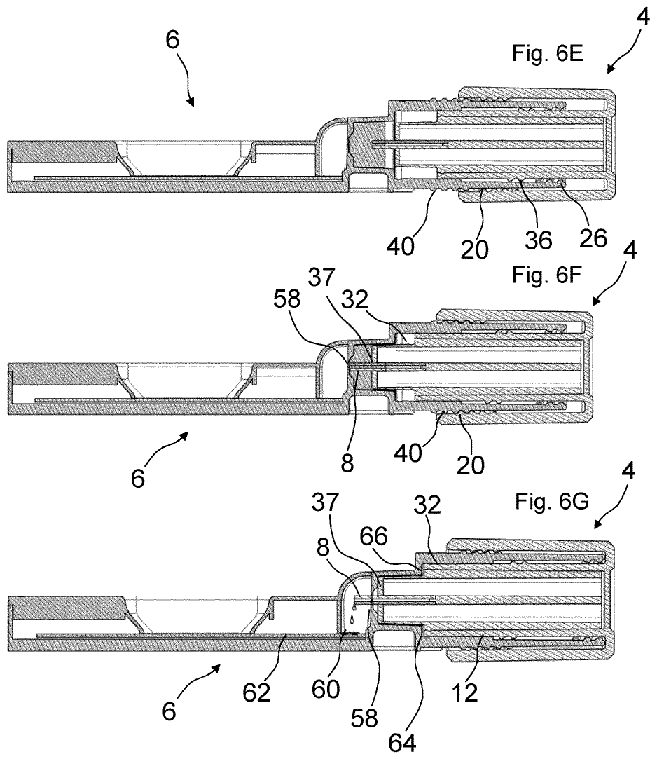

FIGS. 6A-6G illustrate the use sample collection apparatus shown in FIG. 1, specifically showing how the sample collection device is brought together with the sample processing device;

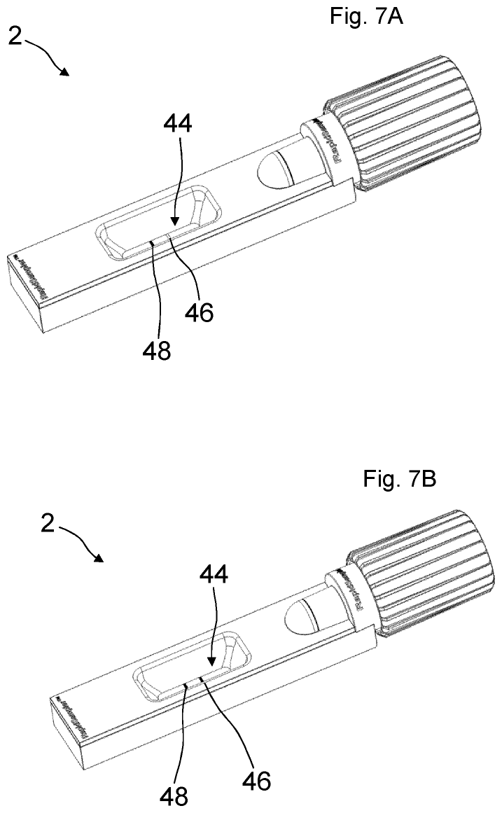

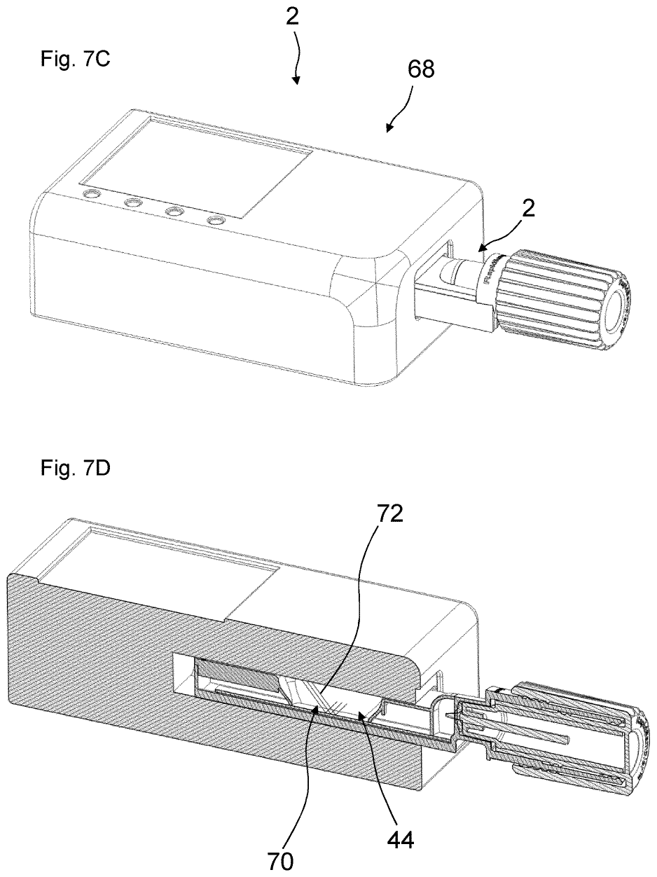

FIGS. 7A and 7B demonstrate how the sample processing device as shown in earlier Figures may be used to perform analysis on the sample;

FIG. 7C shows a perspective view of a sample collection apparatus in accordance with an embodiment of the present invention inserted into an analyser apparatus; and

FIG. 7D shows a cross-sectional view through the sample collection apparatus and analyser apparatus shown in FIG. 7C;

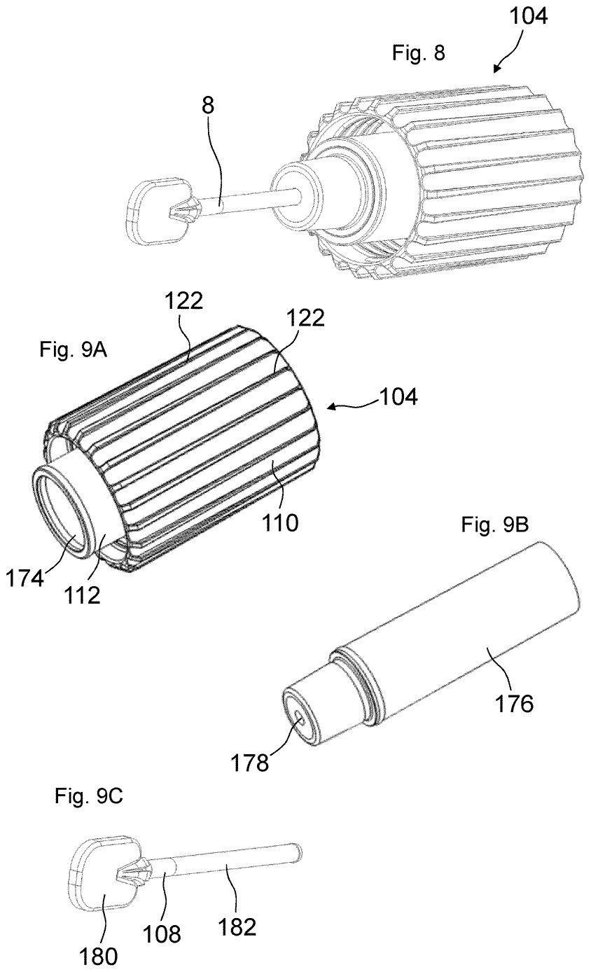

FIG. 8 is a perspective view of a sample collection device in accordance with another embodiment of the present invention, wherein the sample collection element thereof comprises a sample collection patch;

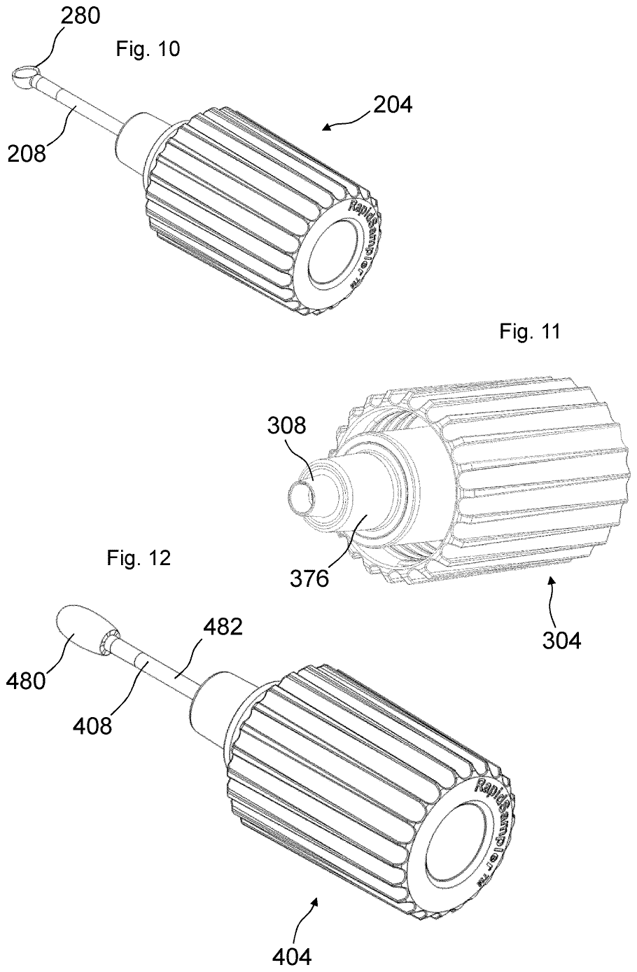

FIG. 9A shows a perspective view of a main body of the sample collection device;

FIG. 9B shows a perspective view of a sample collection element holder;

FIG. 9C shows a perspective view of a sample collection element in the form of a sample collection patch;

FIG. 10 shows a perspective view of a sample collection device in accordance with another embodiment of the present invention, wherein the sample collection element thereof comprises a curette;

FIG. 11 shows a perspective view of a sample collection device in accordance with another embodiment of the present invention, wherein the sample collection element thereof comprises a sample collection tube;

FIG. 12 shows a perspective view of a sample collection device in accordance with another embodiment of the present invention, wherein the sample collection element thereof comprises a swab;

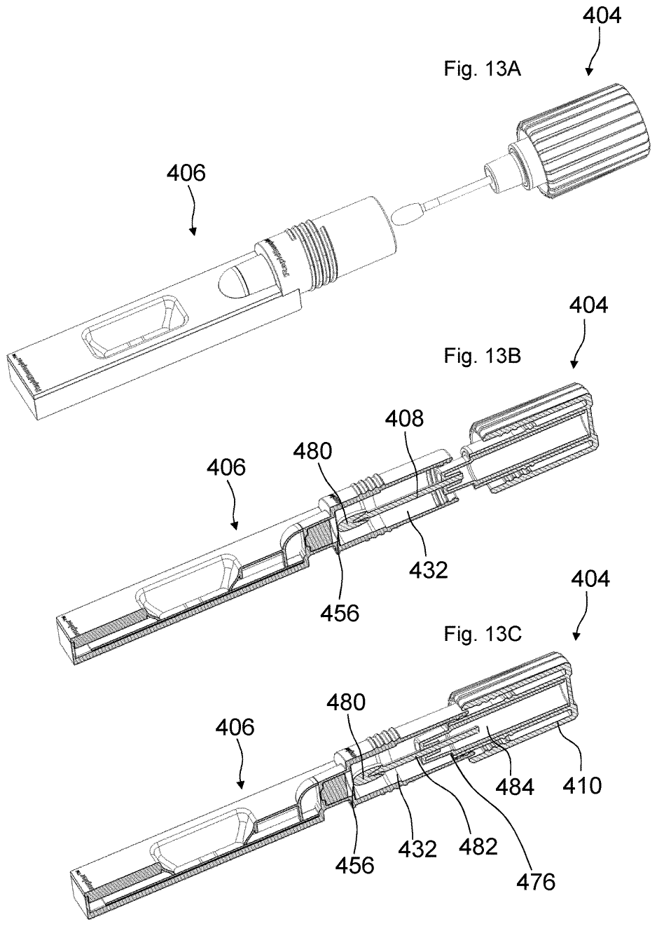

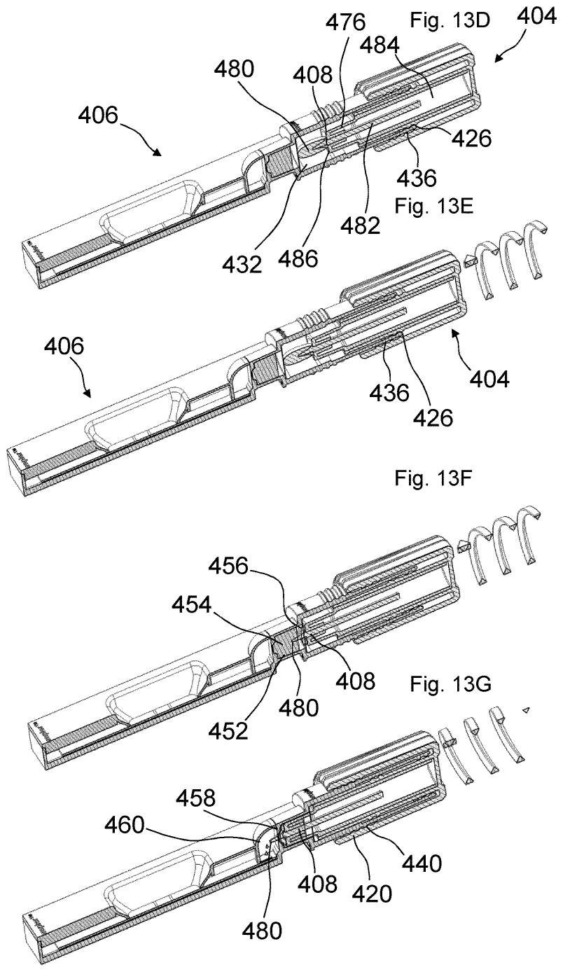

FIGS. 13A-13G illustrate the insertion of a sample collection device comprising a sample collection element in the form of a sample collection patch into a sample processing device, in accordance with an embodiment of the present invention, demonstrating how the sample collection element may partially retract into a main body of the sample collection device during insertion into the sample processing device;

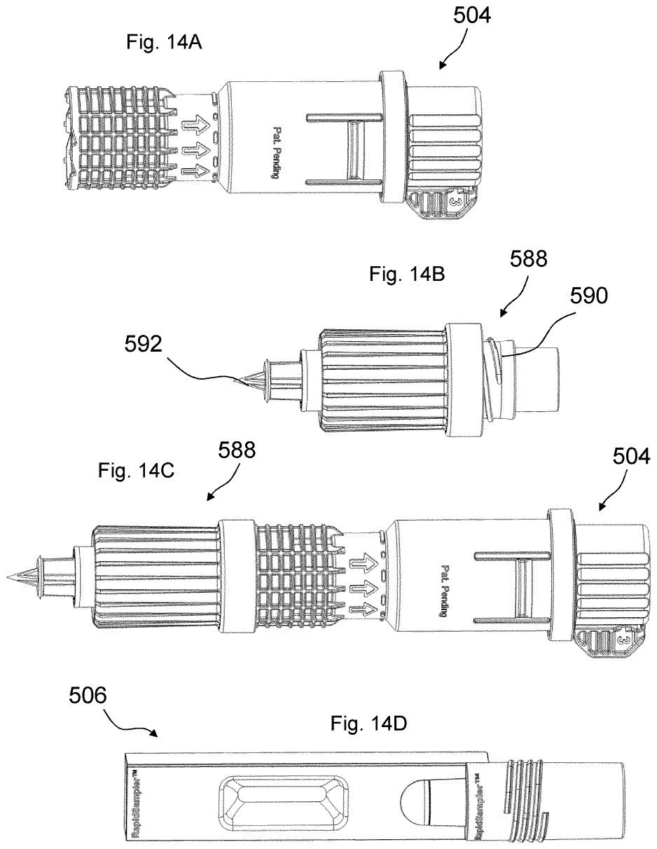

FIGS. 14A-14D illustrate the parts of a sampling apparatus according to an embodiment of the present invention;

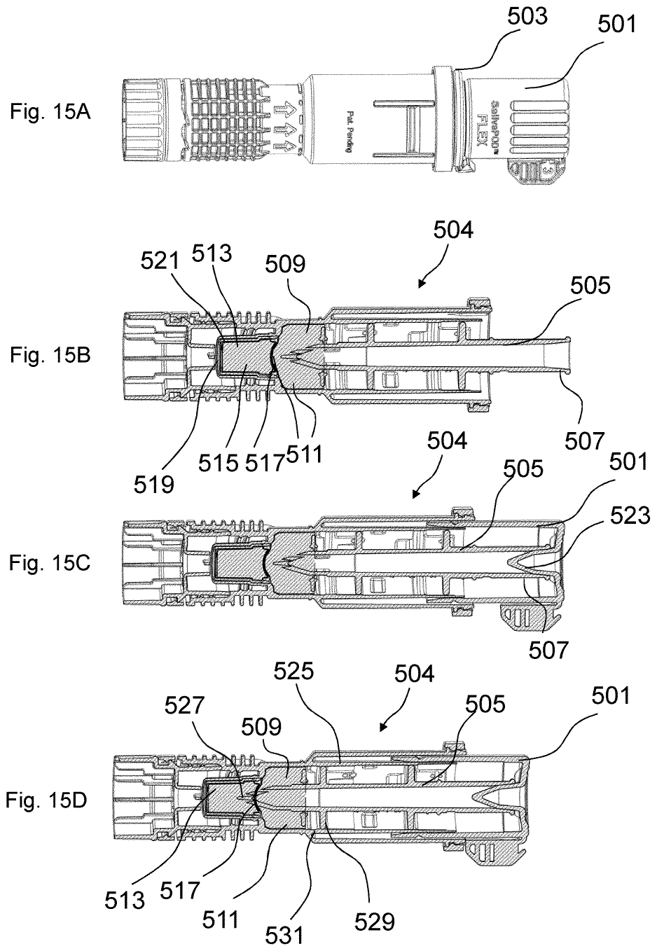

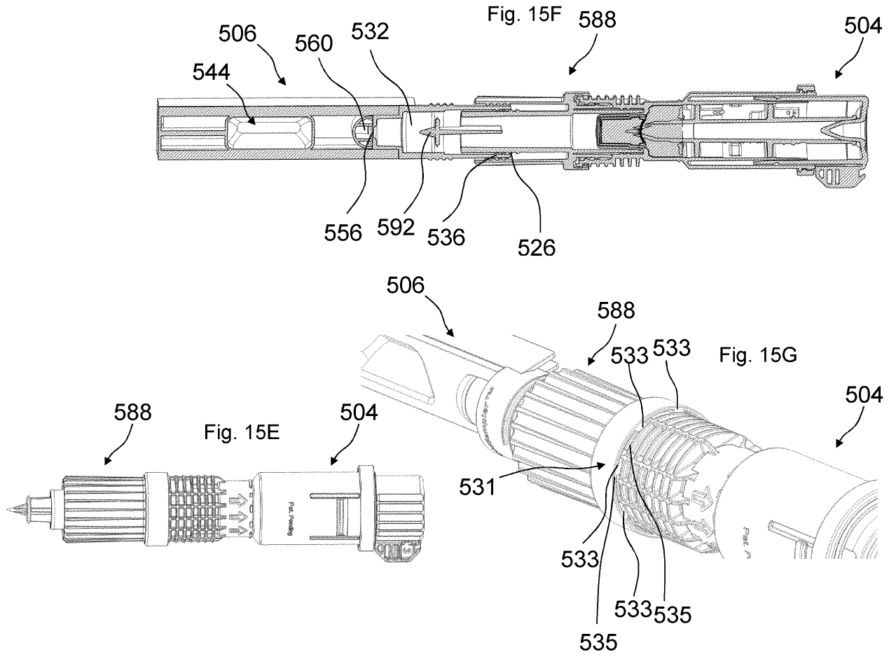

FIGS. 15A-15I illustrate the sampling apparatus shown in FIGS. 14-14D in operation;

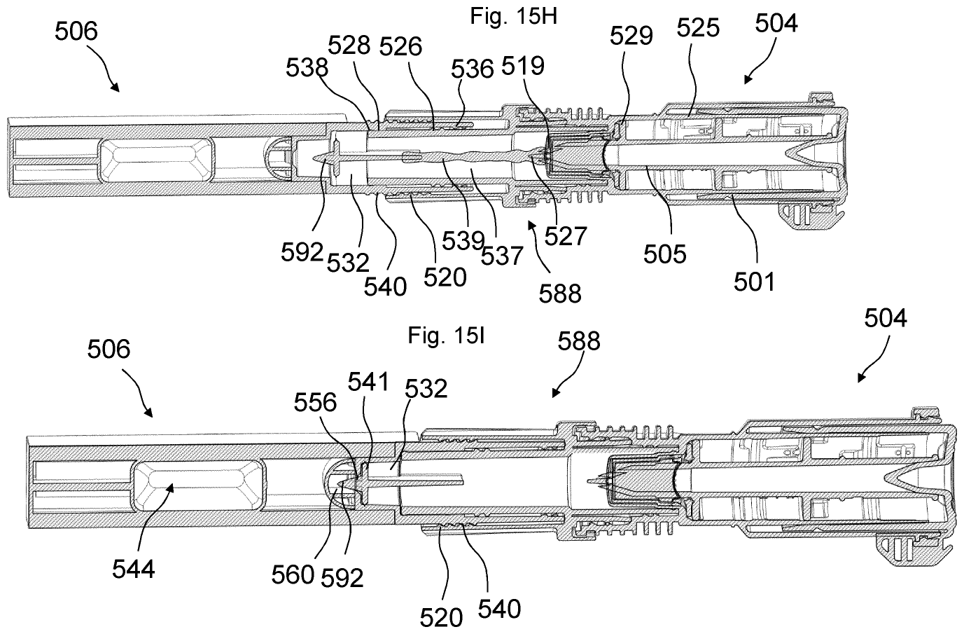

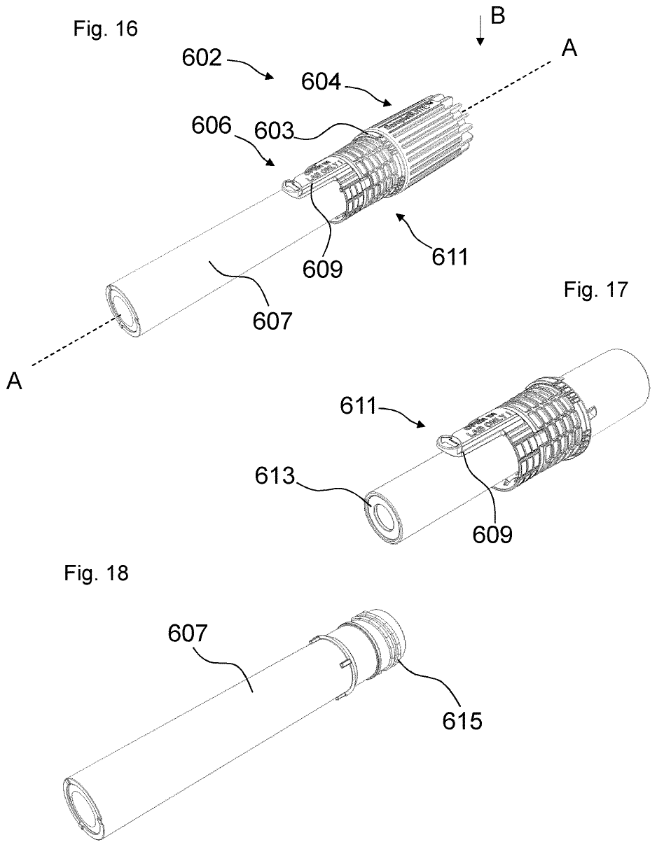

FIG. 16 shows a perspective view of another embodiment of a sampling apparatus;

FIG. 17 shows a perspective view of the housing of the sampling apparatus shown in FIG. 17, with the sample collection device and tube removed;

FIG. 18 shows a perspective view of the tube of the sampling apparatus shown in FIG. 16;

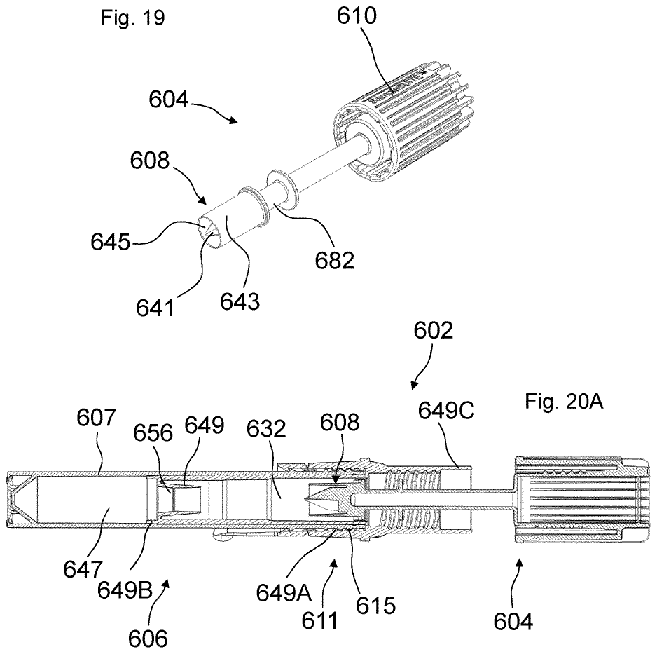

FIG. 19 shows perspective view of the sample collection device shown in the sampling apparatus of FIG. 16;

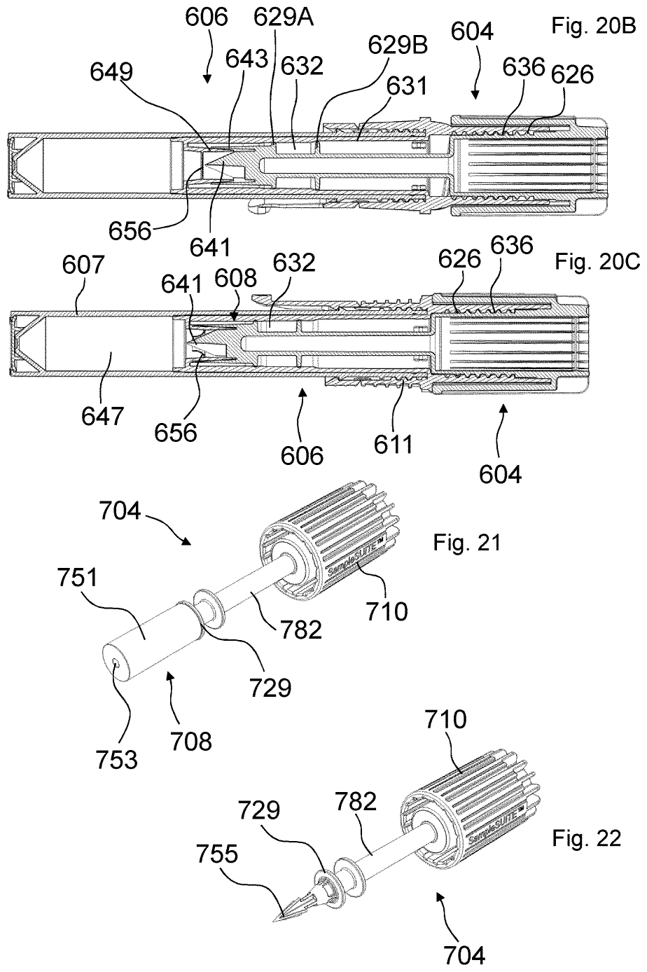

FIGS. 20A-20C show cross-sectional views through the sampling apparatus shown in FIG. 16 as the sample collection device is being inserted into the sample processing device;

FIG. 21 shows a perspective view of an embodiment of a sample collection device comprising an absorbent body;

FIG. 22 shows a perspective view of the sample collection device shown in FIG. 21 with the absorbent body removed;

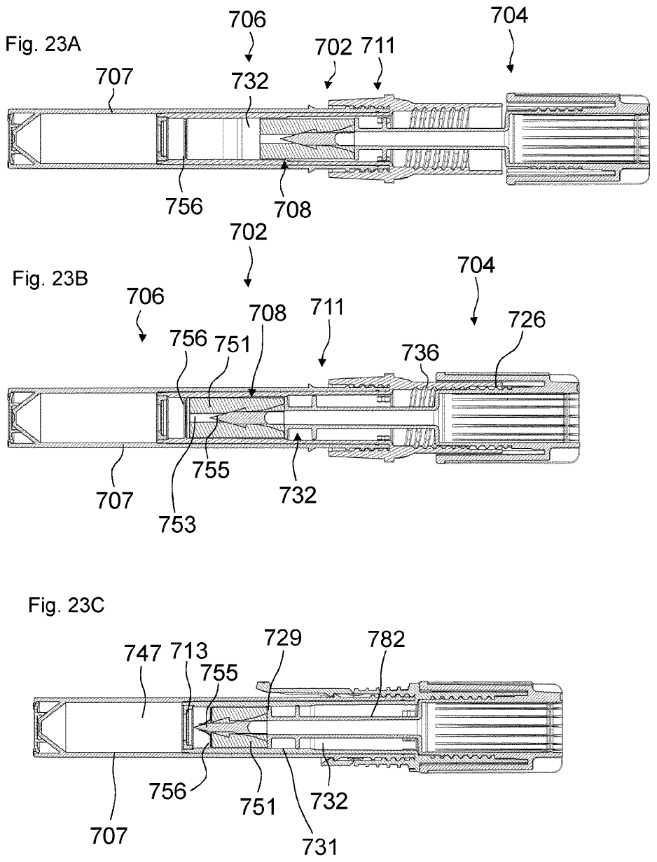

FIG. 23A-23C show cross-sectional views through a sampling apparatus as the sample collection device shown in FIG. 21 is inserted into the sample processing device;

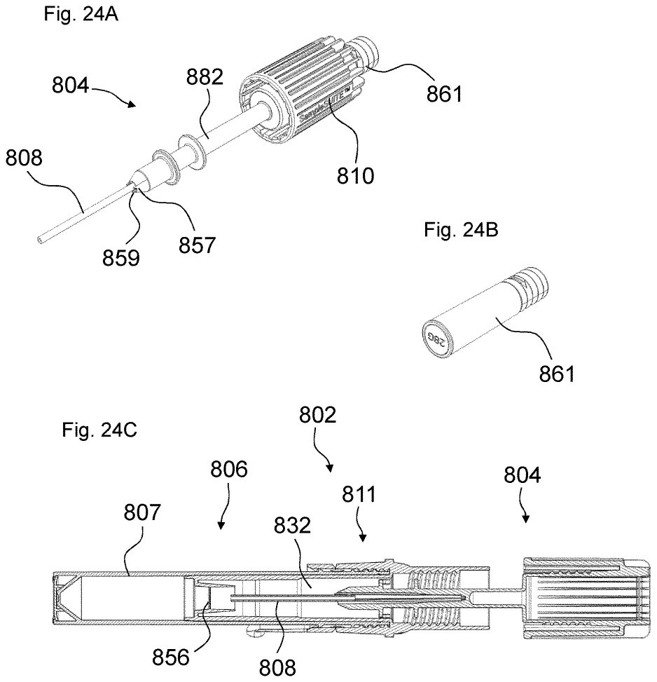

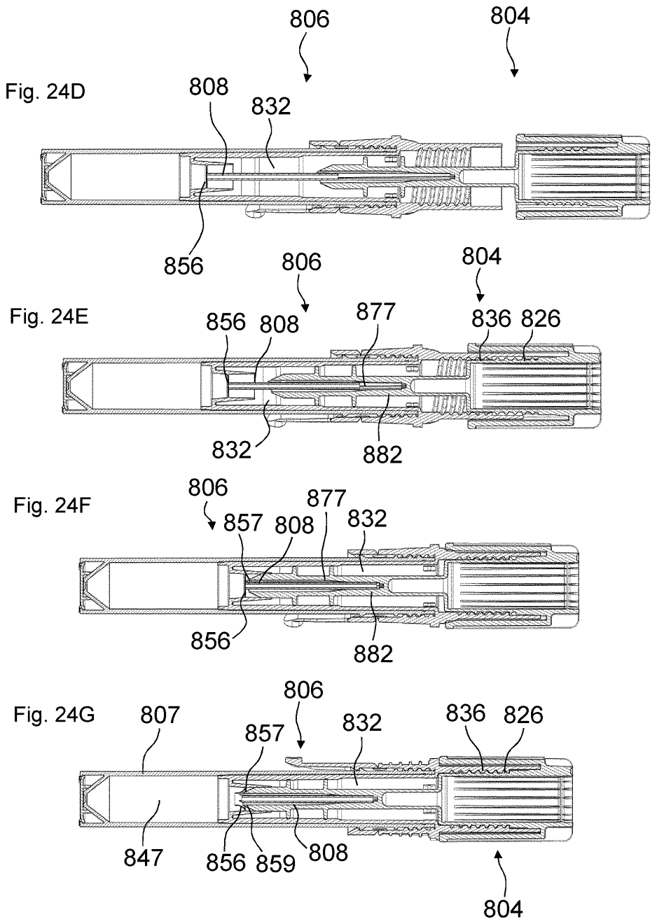

FIG. 24A shows a perspective view of an embodiment of a sample collection device comprising a capillary tube;

FIG. 24B shows a perspective view of the retractable puncture element used on the device shown in FIG. 24A;

FIGS. 24C-24G shows cross-sectional views of a sampling apparatus with the sample collection device shown in FIG. 24A being inserted therein;

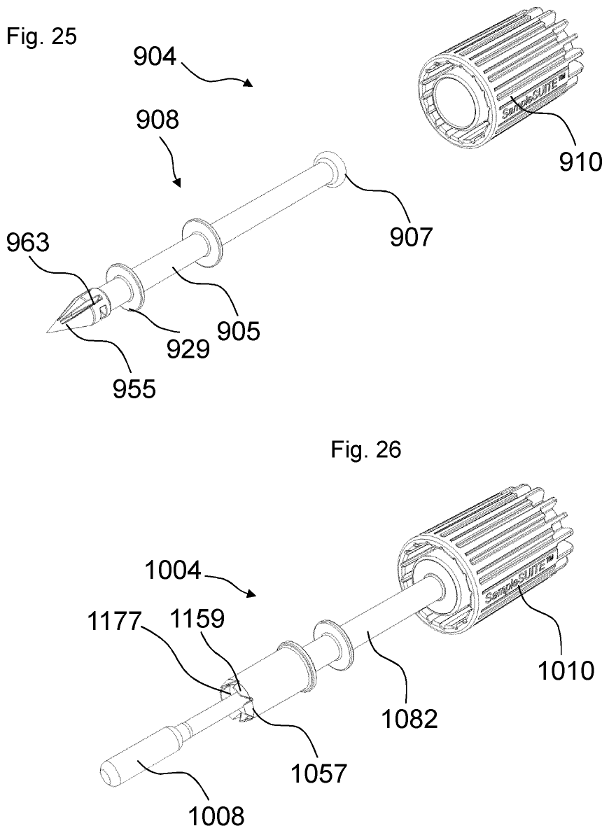

FIG. 25 shows a perspective view of an embodiment of a sample collection device comprising a sample collection conduit;

FIG. 26 shows a perspective view of an embodiment of a sample collection device comprising a swab;

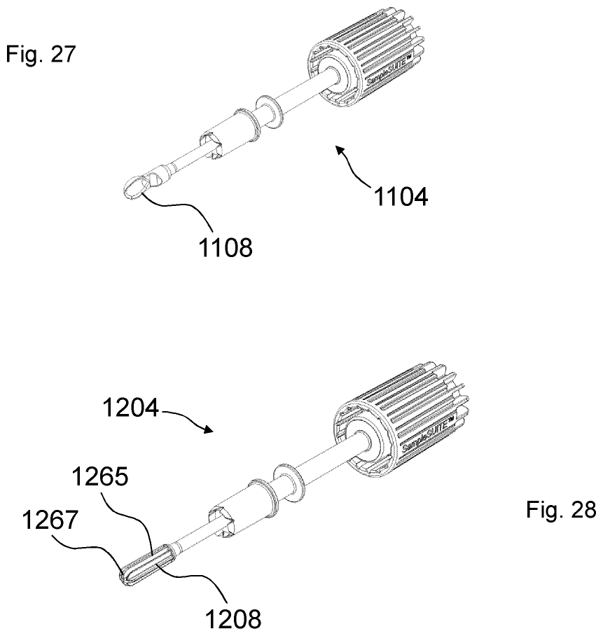

FIG. 27 shows a perspective view of an embodiment of a sample collection device comprising a curette;

FIG. 28 shows a perspective view of an embodiment of a sample collection device comprising a body with a plurality of slots therein to increase surface tension;

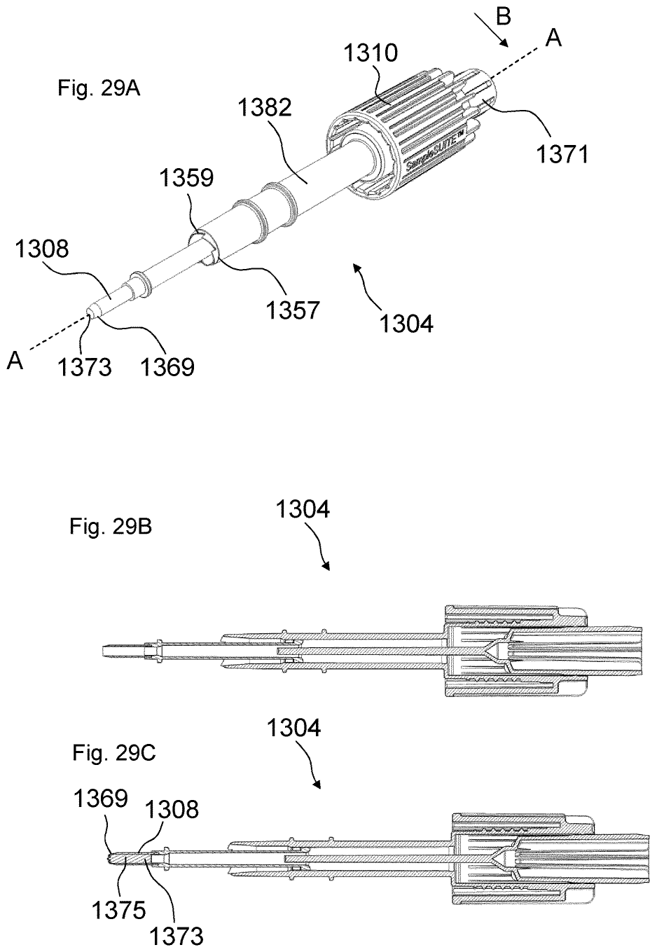

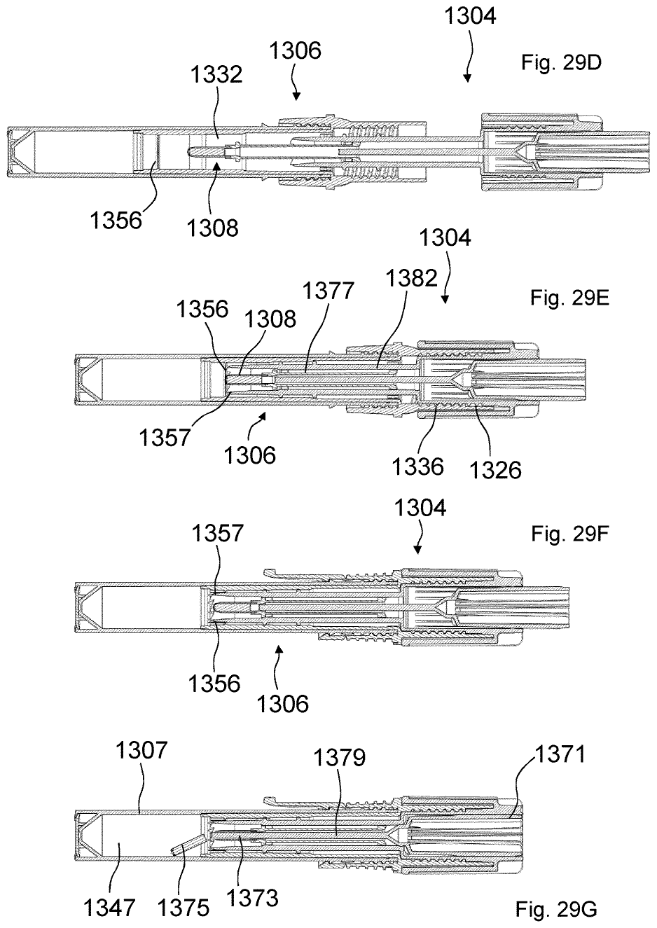

FIG. 29A shows a perspective view of an embodiment of a sample collection device comprising a sharpened element for collecting a sample;

FIGS. 29B-29G show cross-sectional views of the sample collection device shown in FIG. 29A being used to collect a sample and the sample collection device being inserted into a sample processing device;

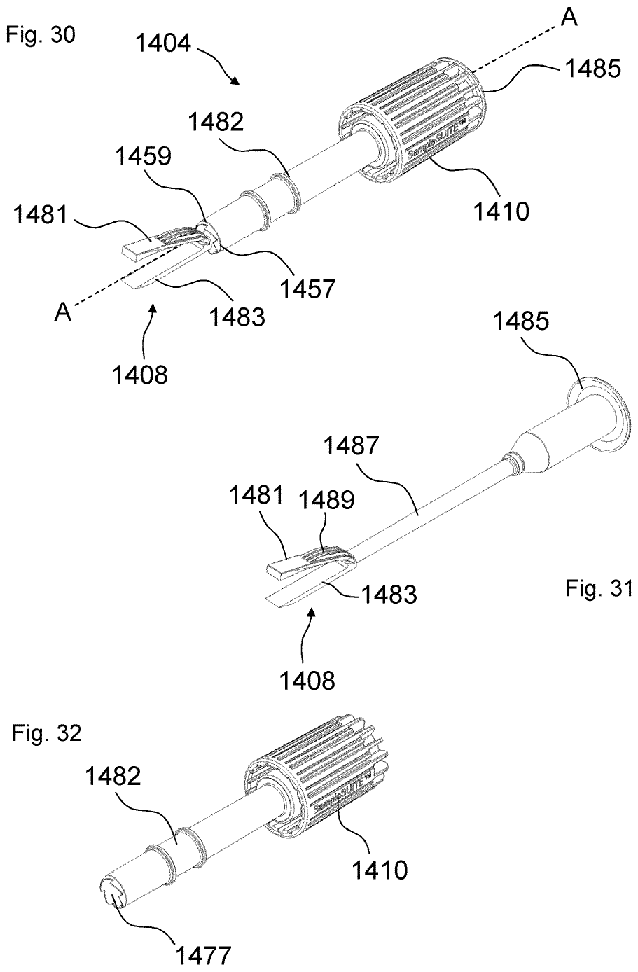

FIG. 30 shows a perspective view of an embodiment of a sample collection device comprising a gripping arrangement;

FIG. 31 shows a perspective view of part of the sample collection device shown in FIG. 30

FIG. 32 shows a perspective view of another part of the sample collection device shown in FIG. 30; and

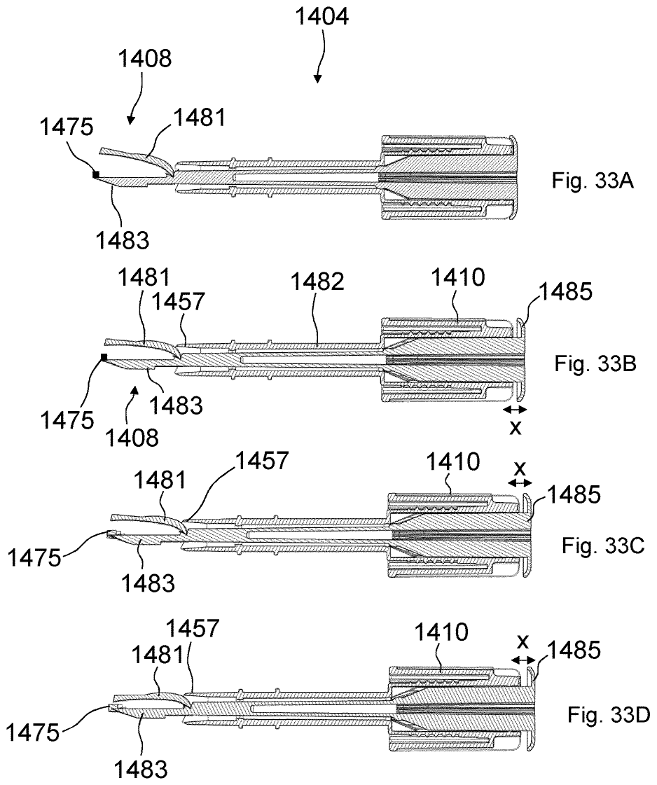

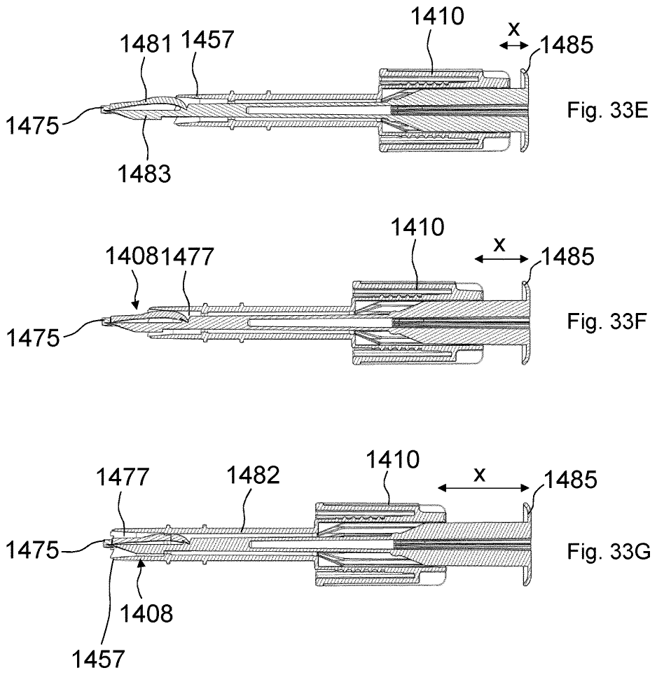

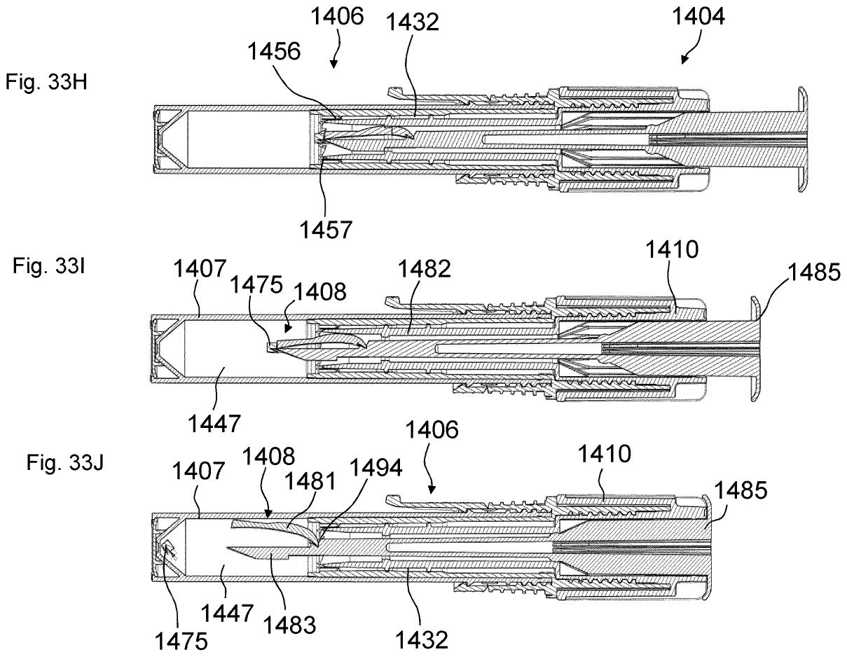

FIGS. 33A-33J shows cross-sectional views of the sample collection device shown in FIG. 30 being used to collect a sample and also show the sample collection device with the sample gripped therein being inserted into a sample processing device.

FIG. 1 shows a perspective view of a sampling apparatus 2 in accordance with an embodiment of the present invention. The sampling apparatus 2 comprises a sample collection device 4 and a sample processing device 6. The sampling apparatus 2 is shown in FIG. 1 with the sample collection device 4 inserted into a cavity (not visible) of the sample processing device 6. However, the sample collection device 4 is separable from the sample processing device. Indeed, the sample collection device 4 may initially be separate from the sample processing device 6 so that a user can proceed immediately to collecting a sample. In other embodiments, the sample collection device 4 may initially be pre-attached to the sample processing device 6. In such embodiments, it may be necessary to first separate the sample collection device 4 from the sample processing device 6 in order to collect a sample. Each of the sample collection device 4 and sample processing device 6 will now be described in isolation with reference to FIGS. 2 to 5.

FIG. 2 shows a perspective view of the sample collection device 4 in isolation, separated from the sample processing device 6. The sample collection device 4 may initially be separate the sample processing device 6 shown in FIG. 1 so as to be capable of collecting a sample. The sample collection device 4 comprises a sample collection element 8. In the embodiment depicted, the sample collection element 8 is in the form of a capillary tube 8 which may be capable of collecting small volumes of liquid samples, e.g. blood. The capillary tube 8 may collect and hold a liquid sample therein under capillary action. Whilst in the embodiment depicted the sample collection element 8 is in the form of a capillary tube, it will be appreciated that the sample collection element 8 may be in any suitable form for collection of the appropriate sample and the features described below in relation to the sample collection device 4 may equally be applied to sample collection devices 4 comprising different sample collection elements.

In some embodiments, as depicted, the sample collection device 4 comprises a main body 10 which comprises a core 12. The core 12 is shaped to receive a holder 14 which itself receives the sample collection element 8. The holder 14 may be removable from the core 12 so as to allow different sample collection elements 8 to be inserted therein. However, in alternative embodiments, the sample collection element 8 may be affixed to the main body 10 in a fixed manner and the holder 14 may be omitted.

An annular space 16 is defined between the core 12 and an inner surface 17 of an outer wall 18 of an the main body 10. A fourth threaded portion 20 is arranged on the inner surface 17 of the outer wall 18 of the main body 10.

A plurality of ridges 22 extend axially along an outermost surface of the outer wall 18 of the main body 10. The ridges 22 may improve the grip on the sample collection device 4 by a user. Any suitable means may be provided to improve a user's grip on the device 4.

FIG. 3 shows a cross-sectional view through the sample collection device 4. As shown in this Figure, the fourth threaded portion 20 is arranged on an inward facing surface 24 of the inner wall 18 of the main body 10. The sample collection device 4 may, as depicted, comprise a second threaded portion 26 arranged on an outward facing surface 28 of the core 12 of the sample collection device 4. The holder 14, which functions to hold the sample collection element 8, may be removably held within a space 30 within the core 12.

FIG. 4 shows a perspective view of the sample processing device 6 in isolation from the sample collection device 4. The sample processing device 6 comprises a cavity 32 which receives at least a part of the sample collection device 4 during use. In the embodiment depicted, the cavity 32 is shaped to receive the core 12 of the sample collection device 4, which carries the sample collection element 8. The outer wall 18 of the sample collection device 4 is not received within the cavity 32. However, in other embodiments, the cavity 32 may be shaped to receive any suitable part, or indeed all of, the sample collection device 4.

The cavity 32 may be defined by a cylindrical outer wall 34 thereby forming a substantially cylindrically shaped cavity 32. The outer wall 34 may be a tubular wall. However, it will be appreciated that the cavity 32 may have any suitable shape. The cavity 32 comprises an opening 64 at its forward end for receiving at least part of the sample processing device 4. The sample processing device 6 may comprise a first threaded portion 36 arranged on an inward facing surface 38 of the sample processing device 6, e.g. on an inward facing surface 38 of the cavity 32. The sample processing device 6 may further comprise a third threaded portion 40 arranged on an outward facing surface 42 of the sample collection device 6. As depicted, the first and third threaded portions 36, 40 may be displaced along an axis, illustrated by dashed line A-A of the sample collection device 6. As will be appreciated by those skilled in the art, displacing the first and third threaded portions 36, 40 along the length of the sample processing device 6 may at least partially function to determine the point at which the first and third threaded portions 36, 40 engage their corresponding second and fourth threaded portions 26, 20.

In some embodiments, as depicted, the sample processing device 6 may comprise a sample analysis means 44 arranged therein. In the embodiment depicted, the sample analysis means 44 is in the form of a lateral flow test which comprises a test indicator 46 and a control indicator 48. The test indicator 46 may be configured to indicate the presence of a biomarker within the sample. The sample processing device 6 may comprise a window 50 which allows a user to see the control and test indicators 48, 46, thereby allow a user to observe the outcome of the analysis of the sample analysis means 44. Whilst a sample analysis means 44 in the form of a lateral flow test is depicted, it will be appreciated that any suitable sample analysis means 44 may be included. Indeed, in other embodiments, the sample analysis means 44 may be omitted entirely, and the sampling apparatus 2 may instead only be used for the collection and partial processing of a sample.

FIG. 5 shows a cross-sectional view through the sample processing device 6 shown in FIG. 4. The sample processing device 6 comprises a reagent 54 arranged within the cavity 32. In some embodiments, the cavity 32 may comprise a reagent chamber which 52 contains a reagent 54. The reagent chamber 52 may be a sub-divided portion of the cavity 32. A wall of the reagent chamber 52, which functions to contain the reagent within the reagent chamber 52, may comprise a first breakable seal 56. Further, in some embodiments, a second wall of the reagent chamber 52 may comprises a second breakable seal 58. The other walls of the reagent chamber 52 may be defined by a main body of the sample processing device 6. The first and second breakable seals 56, 58 may be selectively broken, e.g. penetrated, by the sample collection device 4 when it is inserted into the cavity 32. This will be described in more detail below with reference to later Figures. The first and second breakable seals 56, 58 may be walls of a capsule which is arranged within the cavity 32. The reagent chamber 52 may thus be defined by the capsule. As will be appreciated by those skilled in the art, prior to breaking of the first and second breakable seals 56, 58, the reagent 54 may be fully contained within the reagent chamber 52, and no fluid may be able to enter the reagent chamber 52. The first and second breakable seals 56, 58 act to partition the cavity 32 and define a first portion, i.e. the reagent chamber 54.

In some embodiments, as depicted, the sample processing device 6 may comprise a processing chamber 60 which may be arranged adjacent to the second breakable seal 58. The processing chamber 60 may thus be positioned to receive a sample and reagent mixture, once the second breakable seal 58 has been broken. The processing chamber 60 may facilitate further processing of the sample and reagent mixture. Indeed, in some embodiments, as shown in FIG. 5, the sample processing device 6 comprises a sample analysis means 44 in the form of a lateral flow test. In the embodiment depicted, the sample analysis means 44 comprises an absorbent sheet 62 on which the control line 48 and test line 46 are provided. In the embodiment shown the absorbent sheet extends into the processing chamber 60 such that the sample and reagent mixture therein can be absorbed by the absorbent sheet 62 and travel towards the control and test indicators 48, 46.

With reference back to FIGS. 2 to 5, the first and second threaded portions 36, 26 each have a first handedness and form a first thread arrangement. The third and fourth threaded portions 40, 20 each have a second handedness, different to the first handedness, and form a second thread arrangement. As will be appreciated by those skilled in the art, in order to advance in the same linear, e.g. forward direction, during engagement of the first and second threaded portions, rotation in a first rotational direction is required and during engagement of the third and fourth threaded portions, rotation in a second, different rotational direction is required.

Use of the sampling apparatus 2 shown in FIGS. 1 to 5 will now be described with reference to FIGS. 6A to 6G. Each of FIGS. 6A to 6G show a cross-sectional view of the sampling apparatus 2 at different stages of insertion of the sample collection device 4 into the sample processing device 6. Prior to insertion of the sample collection device 4 into the cavity 32 of the sample processing device 6, a sample is first collected using the sample collection device 4 whilst it is separate to the sample processing device 6. In the embodiment depicted whereby the sample collection element 8 of the sample collection device 4 comprises a capillary tube, this may comprise the user placing the sample collection element 8 into a liquid sample, e.g. blood. The liquid sample may then be drawn into and held within the sample collection element 8 under capillary action.

Once the sample has been collected, a user can bring the sample collection device 4 towards the sample processing device 6 ready for processing. This initial stage is illustrated in FIG. 6A whereby the sample collection element 8 of the sample processing device 4 is inserted into the opening 64 of the cavity 32.

As shown in FIG. 6B, as the sample collection device 4 is advanced further into the cavity 32, the first threaded portion 36, on the sample processing device 6, and the second threaded portion 26, on the sample collection device 4, will come into engagement with one another. Once the first and second threaded portions 36, 26 are engaged with one another, advancement of the sample collection device 4, specifically the sample collection element 8, into the cavity 32 along a first (e.g. forward) linear direction is achieved by rotation of the sample collection device 4 relative to the sample processing device 6. The first and second threaded portions 36, 26 have a first handedness such that as the sample collection device 4 is rotated in a first rotational direction, e.g. clockwise, the threaded engagement between the first and second threaded portions 36, 26 will cause the sample processing device 4, e.g. the sample collection element 8 thereof, to advance forward in the cavity 32. Use of the first and second threaded portions 36, 26 as a means for advancing the sample collection device 4 into the cavity 32 may provide for a controlled means for moving the sample collection device 4 relative to the sample processing device 6.

This advancement of the sample collection device 4, specifically the sample collection element 8 thereof, into the cavity 32 is shown in FIG. 6C whereby the sample collection element 8 has progressed within the cavity 32 towards the first breakable seal 56. As visible in this Figure, the first and second threads 36, 26 are still in engagement with one another at a point at which the sample collection element 8 is in contact with the first breakable seal 56. With reference to FIG. 6D, as the sample collection device 4 is rotated further, the progression along the first and second threaded portions 36, 26 will cause the sample collection device 4, in the embodiment depicted specifically the sample collection element 8 thereof, to break the first breakable seal 56. In the embodiment depicted, the sample collection element 8 contacts the first breakable seal 56 and a pressure applied by the sample collection element 8 on the first breakable seal 56 causes the first breakable seal to break. At this point, the sample collection element 8 may extend into the reagent chamber 52 and the sample may be free to mix with the reagent 54 contained within the reagent chamber 52. As discussed previously, containing the reagent within the sample processing device 6 behind a first breakable seal 56 may advantageously ensure that a correct amount of reagent is present for mixing with the sample. Additionally, in breaking the first breakable seal 56 with the sample collection device 4, e.g. the sample collection element 8 thereof, unnecessary exposure of the reagent may be avoided. This may help to avoid contamination of the reagent. In the embodiment depicted, the sample collection element 8 itself breaks the first breakable seal 56.

At the point at which the sample collection device 4 breaks the first breakable seal 56, or at a stage before the sample collection device 4 has reached this position, the sample collection device 4 may function to seal the cavity 32 so as to prevent the sample and reagent mixture from escaping from the apparatus 2. In some embodiments, as depicted, the core 12 of the sample collection device 4 may be dimensioned such that its outer surface 28 engages with the inner surface 38 of the cavity 32. This engagement may act to seal the cavity 32 and thereby prevent the sample and reagent mixture from escaping. This may help to avoid contamination of the sample and reagent mixture, as well as avoiding contamination of the local environment by the sample and reagent mixture. In sealing the cavity, the sample and reagent mixture may safely be stored within the apparatus 2 for a period of time. With the cavity 32 sealed, a user may also be free to shake the apparatus 2, so as to facilitate mixing of the sample and reagent, without risk of the sample and reagent escaping the cavity 32. Whilst in the embodiment depicted, the cavity 32 is sealed by engagement of the outer wall 38 and inner wall 28, it will be appreciated that sealing of the cavity 32 may be achieved by any suitable means.

As shown in FIG. 6E, the sample collection device 4 may be rotated to a point at which the first and second threaded portions 36, 26 are no longer engaged with one another. In this case, the first and second threaded portions 36, 26 may be open ended such that they can disengage from one another. The first and second threaded portions 36, 26 may be configured such that once the sample collection device 4 has reached this position whereby they are no longer engaged, it may no longer be possible to separate the sample collection device 4 from the sample processing device 6. At least one of the first and second threaded portions 36, 26 may comprise a movement retraction means, e.g. in the form of a ratchet which prevents the first and second threaded portions 36, 26 from being rotated in reverse. Such a movement retraction means may comprise at least one ratchet feature arranged on the sample processing device 6, arranged to engage with a corresponding ratchet feature on the sample collection device 4. The ratchet features may prevent rotation of the sample collection device 4 backwards relative to the sample processing device 6 in a direction which separates the sample collection device 4 from the sample processing device 6. Such ratchet features may be provided at any suitable place on the sample collection device 4 and sample processing device 6, e.g. within the first and second threaded portions 36, 26 thereof.

At the point at which the first and second threaded portions 36, 26 become disengaged, as shown in FIG. 6E, the third and fourth threaded portions 40, 20 are not engaged with one another. So as to engage the third and fourth threaded portions 40, 20, a user may push the sample collection device 4 relative to the sample processing device 6 to a point at which the third and fourth threaded portions 40, 20 are engaged. In other embodiments, the third and fourth threaded portions 40, 20 may be arranged such that they become engaged as soon as the first and second threaded 36, 26 have become disengaged.

As discussed previously, the third and fourth threaded portions 40, 20 have the opposite handedness to the first and second threaded portions 36, 26. As such, in order to advance the sample processing device 4 in the same forward linear direction into the cavity 32, the sample processing device 4 has to be rotated in the opposite direction (e.g. counter-clockwise), to the direction in which it is rotated during engagement of the first and second threaded portions 36, 26.

As shown in FIG. 6E, the sample collection device 4 comprises a plunger 37 at its forward end. The plunger 37 functions to force the sample and reagent mixture out of the cavity 32 as the sample collection device 4 is advanced relative to the sample processing device 6. As depicted, in the embodiment shown, the plunger 37 is shaped such that it only begins to operate, i.e. to apply a force to the liquid within the cavity 32, once the first breakable seal 56 has been broken. This is achieved by appropriate shaping of the plunger 37 relative to the internal dimensions of the cavity 32.

As shown in FIG. 6F, once the third and fourth threaded portions 40, 20 are engaged, rotation of the sample collection device 4 relative to the sample processing device 6 around the second rotational direction (opposite to the first rotational direction) will further advance the sample collection device in a forward linear direction within the cavity 32 of the sample processing device 32. In the embodiment shown, this will cause the sample collection element 8 to progress towards the second breakable seal 58. As the sample collection device 4 is rotated further, the sample collection element 8 will penetrate the second breakable seal 58. This is shown in FIG. 6G whereby the sample collection element 8 has penetrated the second breakable seal 58, thereby allowing the sample and reagent mixture to pass into the processing chamber 60. At this point, a forward end 64 of the core 12 of the sample collection device 4 abuts against an inner rim 66 of the cavity 32 and further linear movement of the sample collection device 4 into the sample processing device 6 is prevented.

The opposite handedness of the third and fourth threaded portions 40, 20 means that following rotation around a first, e.g. clockwise direction, to advance the sample collection device 4 into the first position whereby the first breakable seal 56 is broken, in order to advance the sample collection device 4 further so as to penetrate the second breakable seal 58, a user has to consciously reverse the direction of rotation of the sample processing device 4 so as to advance through the third and fourth threaded portions 40, 20. As such, the chance of a user inadvertently penetrating both the first and second breakable seals 56, 58 may be reduced. The plunger 37 on the forward end of the sample collection device 4 functions to expel the sample and reagent mixture into the processing chamber 60. Of course, in other embodiments, the sample and reagent mixture may pass into the processing chamber 60 by other means, e.g. under the action of gravity.

Once the sample and reagent mixture has passed into the processing chamber 60, the sample and reagent may be analysed by the sample analysis means 44. For example, the absorbent sheet 62 may absorb at least a portion of the sample and reagent mixture and allow said mixture to progress towards the control and test line 48, 46.

In the embodiment described above, the sample collection element 8 functions to break the first and second breakable seals 56, 58. However, any other suitable part of the sample collection device 4 may function to break the first and second breakable seals 56, 58. Indeed, in some embodiments, the sample collection device 4 may act on an intermediate part, e.g. a part of the sample processing device 6, which acts to break the first and second breakable seals 56, 58.

Whilst it has been described above that the sample collection device 4 is rotated relative to the sample processing device 6, it will be appreciated that the sample processing device 6 may instead be rotated relative to the sample collection device 4. Similarly, both the sample collection device 4 and the sample processing device 6 may be rotated with respect to one another.

Whilst the first, second, third and fourth threaded portions 36, 26, 40, 20 facilitate advancement of the sample collection device 4 in a single linear (e.g. forward) direction, using two different directions of relative rotation, with respect to a sample processing device 6, the Applicant has recognised that this arrangement can be can be applied to any two bodies of any medical device whereby control over movement of one body relative to the other is desired. As such, the sample collection device 4 may be considered to be a first body and the sample processing device 6 may be considered to be a second body. The first and second threaded portions 36, 26 may form a first thread arrangement having a first handedness and the third and fourth threaded portions 40, 20 may form a second thread arrangement having a second, opposite, handedness.

FIG. 7A shows the sampling apparatus 2 as shown in previous Figures once the sample has been provided, mixed with the reagent and passed into the chamber 60 for analysis by the sample analysis means 44. As illustrated in FIG. 7A, the control indicator 48 has changed colour, indicating that the sample and reagent mixture has suitably mixed and/or that a sufficient volume of sample and reagent mixture has been provided and/or that the correct type of sample and/or reagent has been provided. In contrast, the test indicator 46 has not yet changed colour. This may indicate that the sample does not contain a particular biomarker.

FIG. 7B shows the sampling apparatus shown in FIG. 7A, whereby both the control and test indicator 48, 46 have changed colour. This may indicate that the sample contains a particular biomarker. In the exemplary case of testing for SARS-COV-2, this may indicate that the provider of the sample has SARS-COV-2.

In the embodiments described above, it may be possible to determine the result of the sample analysis means 44 by observing the changing of colours of indicator lines 46, 48. However, sometimes it may be difficult to observe the change in colour, particularly when the change in colour is relatively faint. Accordingly, as shown in FIG. 7C, the sampling apparatus 2 may further comprise an electronic analysis device 68 into which at least the processing device 6 is inserted. The electronic analysis device 68 may function to read an output of the sample analysis means 44 provided with the processing device 6. As shown in FIG. 7D, the electronic analysis device 68 may comprise a readout means 70, e.g. comprising at least one laser 72 which inspects the sample analysis means 44. The readout means 70 may be capable of more sensitively inspecting the sample analysis means 44 and thereby be capable of detecting even faint control and indicator lines 48, 46.

The Applicant has recognised that the electronic analysis device 68 need not only be suitable for use with a sample analysis means 44 in the form of a lateral flow test as set out above. In other embodiments, the electronic analysis means 68 may be a form of sample analysis means that functions to analyse the sample and reagent mixture. In such embodiments, the sample processing device 6 may not comprise a sample analysis means itself.

The sample collection device 4 may comprise any suitable sample collection element 8 depending on the type of sample which is being collected. FIG. 8 shows another embodiment of a sample collection device 104 which is identical to the sample collection device 4 described above, except that the sample collection element 108 is in the form of a patch collection element 108. The sample collection device 4 may be configured so as to be capable of receiving different sample collection elements, as will be described in more detail below.

FIGS. 9A-9C show the various parts of the sample collection device 104. FIG. 9A shows the main body 110 of the sample collection device 104. As will be appreciated, the main body 110 is shaped to be held by a user. The main body 110 may have a generally cylindrical shape and may comprise a series of ridges 122 extending axially along an outer surface of the main body 110. The ridges 122 may improve a user's grip on the main body 110, which may be particularly useful when rotating the sample collection device 104 relative to a sample processing device into which it is inserted. The main body 110 defines a holder receiving portion 174 which is shaped to receive a holder (shown in FIG. 9B). The holder receiving portion 174 is defined within a core 112 of the sample collection device 104.

FIG. 9B shows a perspective view of a holder 176. The holder 176 comprises a sample collection element receiving portion 178 shaped to receive a sample collection element 108 therein. The holder 176 may be inserted into the holder receiving portion 174 on the main body 110 and may be held therein by a friction fit. In other embodiments, the holder 176 may engage with the holder receiving portion 174 by a threaded engagement or any other suitable arrangement.

FIG. 9C shows a perspective view of a sample collection element 108 in the form of a patch collection element 108. The patch collection element 108 comprises a patch 180 and a rod 182 on which the patch 180 is supported. The rod 182 may be inserted into the sample collection element receiving portion 178 on the holder 176. A number of different sample collection elements 108 may be provided each with a different collection means at an end thereof.

With reference to FIGS. 9A to 9C, the sample collection element 108, specifically the rod 182 thereof may be inserted into the holder 176, specifically into the sample collection element receiving portion 178 thereof, the holder 176 may then be inserted into the holder receiving portion 174 on the main body 110 of the sample collection device 104. In other instances, the holder 176 may be inserted into the holder 176 prior to insertion of the rod 182, of the sample collection element 108, into the sample collection element receiving portion 178. A kit of parts may be provided comprising the main body 110, the holder 176, which defines the sample collection element receiving portion 178 as well as a plurality of different sample collection elements 108. A user may then select the desired sample collection element 108 and insert it into the sample collection element receiving portion 178 ready for use. Each of the sample collection elements 108 may have a rod 182 so as to facilitate insertion of the sample collection element 108 into the sample collection element receiving portion 78. Whilst the sample collection element receiving portion 178 is provided in shown, in the embodiments depicted, in the holder 176, the sample collection element receiving portion 178 may instead be integrally provided with the main body 110.

FIG. 10 shows a perspective view of a sample collection device 204 which is substantially identical to the sample collection device shown in FIG. 8, except that the sample collection element 208 is in the form of a curette which comprises a small scoop 280 at an end thereof for collecting a sample.

FIG. 11 shows a perspective view of another sample collection device 304 which is substantially identical to the sample collection device shown in FIG. 8, except that the sample collection element 308 is in the form of a sample collection tube 308. The sample collection tube 308 may be suitable for collecting any suitable liquid sample, e.g. saliva, urine etc. Whilst visible in this Figure, the sample collection tube 308 may comprise a rod-shaped portion which extends into the holder 376.

FIG. 12 shows a perspective view of another sample collection device 404 which is substantially identical to the sample collection device shown in FIG. 8, except that the sample collection element 408 is in the form of a sample collection swab 408 which comprises a swab 480 at the end of the rod 482.

The main body of each of the sample collection devices 4, 104, 204, 304, 404 may be identical, and simply a different sample collection element may be inserted therein. In other embodiments, each of the sample collection devices 4, 104, 204, 304, 404 comprising their respective sample collection element 8, 108, 208, 308, 408 may be a purpose made sample collection device 4, 104, 204, 304, 404, whereby the sample collection element 8, 108, 208, 308, 408 is permanently fixed in place.

In some embodiments, particularly in embodiments wherein the sample collective device is capable of receiving a number of different sample collection elements, the sample collection element may be capable of moving within the sample collection device. This may allow the sample collection element to move to a position whereby it protrudes from a main body of the sample collection device by a fixed amount. The devices shown in FIGS. 8 to 12 may be capable of facilitating such movement of the sample collection element. Movement of a sample collection element with respect to the sample collection device will now be described with reference to the sample collection device 404 shown in FIG. 12 and further with reference to FIGS. 13A to 13G.

With reference to FIG. 12, a user may first collect a sample using the sample collection device 404. A user may, for example, place the sample collection element 408, specifically the swab 480 into their nasal passage so as to collect a nasal sample. Of course, any other suitable sample may be collected.

The sample collection device 404 may then be brought towards a sample processing device 406, as depicted in FIG. 13A which shows a perspective view of the sample collection device 404 being brought towards a sample processing deice 406. The sample processing device 406 may be identical to the sample processing device 6 described above.

As shown in FIG. 13B, a user may insert the sample collection element 408 into the cavity 432 of the sample processing device 406 to the point at which the swab 480 abuts against the first breakable seal 456.

With reference to FIG. 13C, as a user continues to push the sample collection device 404 into the cavity 432, the first breakable seal 456 may be sufficiently strong that it initially resists penetration thereof and the sample collection element 408, specifically the rod 482 thereof is pushed back into the holder 476. In this regard, the sample collection element 408 may thus be able to move relative to the holder 476, and the holder 476 may comprise an accommodation space 484 arranged to accommodate the rod 482 as it is pushed into the holder 476. Of course, the holder 476 may be omitted and the accommodation space 484 may be integrally formed with the main body 410 of the sample collection device 404. Whilst in the embodiment depicted the swab abuts the first breakable seal 56 which initially resists penetration and forces the rod 482 into the accommodation space 484, other embodiments are envisaged. Any other part may act on the swab 480, or indeed the rod 482 itself, in order to push the rod 482 into the accommodating space 484.

As shown in FIG. 13D, as the sample collection device 404 is pushed into the cavity 432 to the point at which the first and second threaded portions 436, 426 engage with one another, the sample collection element 408 fully retracts into the accommodation space 484 such that only the swab 480 extends therefrom, and the rod 482 is substantially contained within the holder accommodation space 484. The result of this is that the portion of the sample collection element 408, i.e. the swab 480, which extends from a reference point on the sample collection device 404, e.g. from the forward edge 486 of the holder 476, is reduced. The sample collection device 404, and a plurality of sample collection elements, may be configured such that irrespective of which sample collection element is inserted into the sample collection device 404, when advanced into the position shown in FIG. 13D, the length of the sample collection element 408 extending from the reference point, e.g. the forward edge 486 of the holder 476, is the same. As such, the sample collection device 404, irrespective of the sample collection element inserted therein, may be suitable for use with a single sample processing device 106. By ensuring that all the sample collection elements 408 extend by a fixed amount form the reference point, it may ensure that the sample collection element 408 is capable of penetrating the first breakable seal 456, and doing so at the appropriate time, e.g. when the cavity 432 has been suitably sealed.

Once the sample collection element 408 has been retracted into the holder 476 as shown in FIG. 13D, the sampling apparatus 402 comprising the sample collection device 404 and sample processing device 406 operates in a similar manner to the sampling apparatus 2 described above. For completeness, this process is now described and shown in FIGS. 13E to 13G. As shown in FIG. 13E, once the sample collection device 404 has been fully pushed into the sample processing device 406, the first threaded portion 436 and second threaded portion 426, which have a first handedness, are engaged and a user rotates the sample collection device 404 about a first direction (e.g. a clockwise direction).

This rotational movement further advances the sample collection device 404 into the sample processing device 406. As shown in FIG. 13F, this advancement will eventually cause the sample collection element 408, specifically the swab 480 thereof, to penetrate the first breakable seal 456. At this point, any sample on the swab 480 will be free to mix with the reagent 456 contained within the reagent chamber 452. After the sample has been mixed with the reagent 454, the sample collection device 404 may be advanced further into the sample processing device 406.

This is shown in FIG. 13G whereby the third threaded portion 440 and fourth threaded portion 420 are engaged. As the third and fourth threaded portions 440, 420 have the opposite handedness to the first and second threaded portions 436, 426, rotation of the sample collection device 404 in the opposite, e.g. counter-clockwise direction, also advances the sample collection device 404 in the same forward linear direction further into the sample processing deice 406, to the point at which the sample collection element 408, e.g. the swab 480 thereof, penetrates the second breakable seal 458, thereby allow the sample and reagent mixture to pass into the processing chamber 460. Any suitable analysis may then be performed on the sample and reagent mixture.

FIG. 14A shows a sample collection device 504 in accordance with another embodiment of the present invention. Features of the sample collection device 504 will be described in more detail later below in relation to FIG. 15A. FIG. 14B shows an adaptor 588 configured to be attached to the sample collection device 504. The adaptor 588 comprises a connection thread 590 arranged to engage a corresponding connection thread on the sample collection device 504 so as to attach the adaptor 588 to the sample collection device 504. The adaptor 588 further comprises a seal breaking element 592 for breaking a seal within the sample processing device. Further features of the adaptor 588 will be described further below with reference to later Figures.

FIG. 14C shows the sample collection device 504 and adaptor 588 attached together. The adaptor 588 may be attached to the sample collection device 504 before, or after, the sample collection device 504 has been used to collect a sample. FIG. 14D shows a sample processing device 506. The sample processing device 506 is largely the same as the sample processing device 6 described above, except that it does not comprise a second penetrable seal and there is no reagent contained within the sample processing device 506. Instead, the reagent is contained within the sample collection device 504 as will be described further below. Whilst the adaptor 588 is shown as being attached to the sample collection device 504, it may be possible to first attach the adaptor 588 to the sample processing device 506, and then attach the sample collection device 504 to the adaptor 588.

Operation of the sampling apparatus depicted in FIGS. 14A-14D will now be described with further reference to FIGS. 15A-15I. With reference to FIG. 15A a sample is first collected using the sample collection device 504. In order to collect a sample, a lid 501 may first be removed. In order to remove the lid 501, it may be necessary to first remove the tamper element 503, which is configured such that it cannot be replaced back on the sample collection device 504 once removed. The tamper element 503 thus indicates that the sample collection device 504 has at least been opened and thus may prevent accidental re-use of the sample collection device 504. In some embodiments, removal of the lid 501 may function to remove the tamper element 503.

FIG. 15B shows the sample collection device 504, in cross-section, with the lid 501 removed. As visible in this Figure, when the lid 501 is removed, a sample collection element in the form of a sample collection conduit 505 is exposed. In order to provide a sample, a user may, for example, place an inlet 507 of the sample collection conduit 505 into their mouth before then spitting saliva into the sample collection conduit 505. The sample 509 may then collect in a sample collection chamber 511 arranged within the sample collection device 504. As depicted in this Figure, the sample collection device 504 may comprise a reagent 513 contained within a reagent chamber 515. The reagent 513 may be arranged within a capsule 521 which can be selectively inserted into the reagent chamber 515. The capsule 521 may comprise a first reagent seal 517 and a second reagent seal 519. At the stage shown in FIG. 15B, the sample 509 and the reagent 513 have not yet mixed.

FIG. 15C shows the sample collection device 504 at a stage at which the lid 501 has been reattached to the sample collection device 504. At this point, a sealing portion 523 of the lid 501 acts to seal the inlet 507 of the sample collection conduit 505. With the lid 501 reattached to the sample collection device 504, the sample collection conduit 505 may be advanced within the sample collection device 504. This is depicted in FIG. 15D. The lid 501 may be advanced relative to a main body 525 of the sample collection device 504. As the lid 501 is advanced, this forces the sample collection conduit 505 through the sample collection chamber 511. A tip 527, which may be pointed and/or sharpened, of the sample collection conduit 505 may contact and break the first reagent seal 517 thereby allowing the sample 509 and reagent 513 to mix with one another. In the embodiment depicted, in at least the position shown in FIG. 15D, a plunger 529, which is coupled to the sample collection conduit 505, seals against an inner wall 531 of the main body 525. This acts to seal the sample 509 and thus the reagent 511 within the sample collection device 504. As such, it may be possible to shake the sample collection device 504 so as to properly mix the sample 509 and the reagent 513.

With reference to FIG. 15E, the adaptor 588 may then be attached to the sample collection device 504. Of course, in some instances, the adaptor 588 may be attached to the sample collection device 504 prior to the collection of a sample. The adaptor 588 may be attached to the sample collection device 504 by screwing the adaptor 588 to the sample collection device 504 so as to engage the connection screw thread 588 (see FIG. 14B) on the adaptor 588, with a corresponding thread on the sample collection device 504. Of course, the adaptor 588 and sample collection device 504 may be engaged and held together via any other suitable means, e.g. a friction fitting or a bayonet-type fitting.

As shown in FIG. 15F, once the adaptor 588 is fitted to the sample collection device 504, the adaptor 588 may be attached to the sample processing device 506. This is achieved by inserting the seal breaking element 592 into the cavity 532 of the sample processing device 506. When the adaptor 588 is attached to the sample collection device 504, the adaptor 588 may be considered to be part of the sample collection device 504. As shown in FIG. 15F, in the embodiment depicted, the sample processing device 506 does not comprise any reagent arranged therein. The sample processing device 506 comprises a first breakable seal 556 which separates the cavity 532 from a processing chamber 560. The sample processing device 506 may comprise a sample analysis mean 544 at least part of which may pass into the processing chamber 560.

As visible in FIG. 15F, the adaptor 588 may comprise a second threaded portion 526 configured to engage with a first threaded portion 536 on the sample processing device 506. The second threaded portion 526 may be configured in an identical manner to the second threaded portion 26 on the sample collection device 4 of the embodiment described above. As such, the first and second threaded portions may have a first handedness such that rotation of the sample collection device 504, together with the adaptor 588, in a first direction (e.g. a clockwise direction) advances the sample collection device 504, specifically the adaptor 588 thereof, into the cavity 532.

FIG. 15G shows a perspective view of the sample collection device 504 with the adaptor 588 attached thereto which in turn is attached to the sample processing device 506. The adaptor 588 comprises a locking means 531 configured to engage with the sample collection device 504 and prevent rotation of the sample collection device 504 relative to the adaptor 588 once the sample collection device 504 and adaptor are fully engaged. In the embodiment depicted, the locking means 531 comprises a plurality of tabs 533 which lock into recesses arranged on the sample collection device 504. Engagement of the tabs 533 with the recesses 535 may prevent rotation of the adaptor 588 and sample collection device 504 relative to one another. Any number of tabs 533 and recesses 535 may be provided. Of course, the locking means 531 may comprise any other suitable arrangement for preventing rotation of the sample collection device 504 relative to the adaptor 588 when in the fully engaged position shown. The locking means 531 may ensure that rotation of the sample collection device 504 results in rotation of the adaptor 588 and thus advancement relative to the sample processing device 506.

With reference back to FIG. 15F, the sample collection device 504 and adaptor 588 may be rotated relative to the sample processing device 506 until the point at which the first and second threaded portions 526, 536 become disengaged. This is shown in FIG. 15H. As visible in FIG. 15H, the adaptor 588 comprises a fourth threaded portion 520 arranged to engage with a third threaded portion 540 provided on the sample processing device 506. However, at the position shown in FIG. 15H, the third and fourth threaded portions 540, 520 have not yet become engaged.

As shown in FIG. 15H, as the adaptor 504 is advanced into the cavity 532 an outward facing surface 528 of the adaptor 588 engages with an inward facing surface 538 of the cavity 532, thereby forming a seal between. This provides a sealed connection between the cavity 532 and a hollow core 537 of the adaptor 588. At this point, a user may dispense the sample and reagent mixture 539 from the sample collection device 504. This may be achieved by further advancing the lid 501 relative to the main body 525 of the sample collection device 504. As visible in FIG. 15H, this acts to drive the sample collection conduit 501 to the point at which the tip 527 of the sample collection conduit 505 breaks the second reagent seal 519. Movement of the sample collection conduit 505 also drives the plunger 529. With the second reagent seal 519 broken, movement of the plunger 529 acts to expel the sample and reagent mixture 539 from the sample collection device 504. As depicted, the expelled sample and reagent mixture 539 passes through the hollow core 537 of the adaptor 588 and into the cavity 532. At this stage, the cavity 532 remains closed and so the sample and reagent mixture 539 may simply be held within the sample processing device 506.

Following the dispensing of the sample and reagent mixture into the sample processing device 506 as described above, further processing may then be performed. With reference to FIG. 15I, the sample collection device 504 and adaptor 588 may be rotated relative to the sample processing device in a second direction, different to the direction during engagement of the first and second threaded portions 536, 526, whilst the third and fourth threaded portions 540, 520 are engaged. As with the embodiment described above, the different handedness of the first and second threaded portions 536, 526 compared to the third and fourth threaded portions 40, 20, ensures that a user has to make a conscious decision to continue progressing the sample collection device 504, and the adaptor 588, relative to the sample processing device 506.

As depicted in FIG. 15I, as the adaptor 588 advances relative to the sample processing device 506 through engagement of the third and fourth threaded portion 540, 520, the seal breaking element 592 will eventually reach a position at which it breaks the first breakable seal 556. Any sample and reagent mixture within the cavity 532 will then be free to flow into the processing chamber 560. In some embodiments, the adaptor 588 may comprise a plunger 541, which may be integrally formed with the sample breaking element 592. The plunger 541 may act to force any sample and reagent mixture out of the cavity 532 and into to the processing chamber 560. As discussed previously, a sample analysis means 544 may be provided, and at least part of the sample analysis means 544 may extend into the processing chamber 560. As such, analysis of the sample and reagent may then be performed.

In the embodiment described above, it is discussed how the sample and reagent are mixed with one another prior to attachment of the sample collection device 504 to the adaptor 588 and subsequently to the sample processing device 506. However, it will be appreciated that the sample collection device 504, adaptor 588 and sample processing device 506 may all be attached together before the sample and reagent are mixed with one another.

The adaptor 588, which comprises the second and fourth threaded portions 26, 20 together with the seal breaking element 592, provides the ability to attach a sample collection device 504 which may otherwise not be intended to operate with the sample processing device 506 and its particular features, i.e. the thread arrangements each having a different handedness. The adaptor 588 may therefore increase the number of sample collection devices that can be used with the sample processing device 506.

FIG. 16 shows a perspective view of a sampling apparatus 602 in accordance with another embodiment. The sampling apparatus 604 comprises a sample collection device 604 and a sample processing device 606. In the embodiment shown, the sample processing device 606 comprises a closure member, in the form of a tube 607, attached to a housing 611. The housing 611 and tube 607 together define the sample processing device 606. A tamper element 609 is provided between the housing 611 and the tube 607. Removal of the tamper element 609 may permit separation of the housing 611 from the tube 607 and provide a visual indication to a user that such a separation has occurred, or at least been initiated. The tube 607 is attached to a distal end of the housing 611. The housing 611, absent the tube 607, may be considered to define an open-end cavity. With the tube 607 attached, a cavity is formed having one end closed by the tube 607. A tamper element 603 may be arranged between the sample collection device 604 and sample processing device 606, in embodiments wherein the sample processing device 604 and sample collection device are pre-attached to one another

FIG. 17 shows a perspective view of the housing 611 in isolation. As shown, the tamper element 609 may form part of, or be attached to, the housing 611. In some embodiments, as shown in FIG. 17, the housing 611 may comprise a filter 613. As depicted, the filter 613 may be arranged at a distal end of the housing 611. The filter 613 may act to filter a sample as it passes through the housing 611 and into the tube 607. The filter 613 may comprise a fine mesh or gauze.

FIG. 18 shows a perspective view of the tube 607 in isolation. The tube 607 comprises a threaded portion 615 which may engage with a corresponding threaded portion on the housing 611. The tube 607 has a closed distal end, such that when the tube 607 is attached to the housing 611 it forms a cavity having a closed end.

FIG. 19 shows a perspective view of the sample collection device 604 in isolation. As depicted, the sample collection device comprises a sample collection element 608. In the embodiment shown in FIG. 19, the sample collection element 608 comprises a tip 641 and a surrounding wall 643. The tip 641 and the surrounding wall 643 together defined a sample collection volume 645 between the surrounding wall 643 and the tip 641. The sample collection device 604 shown in FIG. 19 may be particularly well suited to collecting a solid, or semi-solid, sample, e.g. a stool sample. The sample collection element 608 is connected to a main body 610 of the device 604 by a connecting rod 682. The connecting rod 682 may be solid, or hollow, and have any suitable shape. The main body 610 may define a proximal end of the device 604 and the sample collection element 608 may define a distal end of the device 604. The tip 641, of the sample collection element 608, may protrude further than the surrounding wall 643 and may thus also function as a seal breaking element, as will be described in further detail below. As depicted, in some embodiments, the tip 641 may be pointed and/or sharpened so as to be suitable for breaking the breakable seal. A pointed and/or sharpened tip 641 may also be particularly well suited to penetrating a sample. In use, a user may hold the main body 610 and push the sample collection element 608 into a sample, e.g. a stool sample. The sample will collect in the sample collection volume 645 and when a user subsequently removes the sample collection device 604 from the sample, a portion of the sample will be retained within the sample collection volume 645. A user may then go on to process the sample using the sample processing device 606. This will now be described with reference to FIGS. 20A-20C.

FIGS. 20A-20C show a cross-sectional view through the sampling apparatus shown in FIG. 16 through the line A-A when viewed in the direction indicated by arrow B. As depicted, in FIG. 20A, once a sample has been collected using the sample collection device 604, the user inserts the sample collection device 604, e.g. the sample collection element 608 thereof, into the cavity 632, e.g. into an open proximal end thereof. FIG. 20A shows how the tube 607 is connected to the housing 611 by the threaded portion 615 on the tube 607 and a corresponding threaded portion 649A on the housing 611. As shown in FIG. 20A, the threaded portion 649A on the housing 611 is displaced from the distal end 649B of the housing 611 and is arranged between the distal end 649B and the proximal end 649C of the housing 611. As a result, the distal end 649B of the housing 611 is arranged substantially within the tube 607 when the tube 607 is attached to the housing 611. As depicted in FIG. 20A, the housing 611, of the sample processing device 606, comprises a first breakable seal 656 which defines a first portion 647 of the cavity 632. The first portion 647 of the cavity 632 is closed in this embodiment, between the closed tube 607 and the breakable seal 656. In some embodiments, as depicted, the tube 607 (i.e. the closure member) and the housing 611 together define the cavity 632, which comprises the first portion 647.

The sample housing 611 comprises a projecting wall 649 which projects away from the first breakable seal 656. The projecting wall 649 may have any suitable profile.

As shown in FIG. 20B, a user may continue to advance the sample collection device 604 into the cavity 632 until the point at which a first threaded portion 636 on the housing 611 (of the sample processing device 606) interacts with a second threaded portion 626 on the sample collection device 604. At this point, the sample collection device 604 may be rotated relative to the sample housing 611 in order to advance the sample collection device 604 relative to the sample housing 611, and thus the cavity 632 thereof. As depicted in FIG. 20B, the sample collection device 604 may comprise first and second sealing elements 629A, 629B. When the sample collection device 604 is inserted into the cavity 632 by a sufficient amount, the first and second sealing elements 629A, 629B press against the inner wall 631 of the cavity 632 thereby sealing the cavity 632 upstream of the sample collection element 608. Whilst two sealing elements are depicted, it will be appreciated that only a single sealing element may be included. In some instances, the sealing elements may be omitted entirely.

With continued reference to FIG. 20B, the projecting wall 649, of the housing 611, and the surrounding wall 643 of the sample collection element 608 are dimensioned such that the tip 641 passes into the inside of the projecting wall 649 and the surrounding wall 643 passes around an outer side of the projecting wall 643. This is shown in FIG. 20B. In some embodiments, as depicted, the surrounding wall 643 and projecting wall 649 may be dimensioned such that they contact one another, as shown in FIG. 20B. This may have multiple benefits. For example, the projecting wall 649 may function to separate the sample from the surrounding wall 643, thereby ensuring the sample can be suitably processed. In addition, or alternatively, the surrounding wall 643 and projecting wall 649 may seal against one another, again preventing sample from travelling upstream within the apparatus 602.

With reference to FIG. 20C, further rotation of the sample collection device 604, thereby driving linear movement of the sample collection device 604 relative to the sample processing device 606, through the engagement between the first and second threaded portions 636, 626, causes the tip 641 to break the first breakable seal 656. At this point, the sample contained within the sample collection element 608 is exposed to the first portion 647 of cavity 632 defined between the tube 607 and the housing 611 attached thereto. The first portion 647 may contain a reagent which may then mix with the sample. The sample and reagent may then be stored within the first portion 647 as long as required.

When the sample collection device 604 is attached to the sample processing device 606, once it has reached a particular position, e.g. once the first breakable seal 656 has been broken, the sample collection device 604 may be coupled to the sample processing device 606 in a manner in which rotation of the sample collection device 604 in an opposite direction to the direction in which it is attached, results in rotation of the housing 611 of the sample processing device 606 relative to the tube 607. This coupling may be achieved by a ratchet arrangement acting between the housing 611 and the sample collection device 604 This may allow the tube 607 to be separated from the housing 611 without requiring direct contact with the housing 611. This arrangement may allow separation to be performed by a robotic apparatus more easily. With the tube 607 separated from the housing 611, further processing, e.g. analysis of, the sample may be performed.

FIG. 21 shows a perspective view of a sample collection device 704 in accordance with another embodiment of the present invention. The sample collection device 704 comprises a sample collection element 708 which comprises an absorbent body 751 (e.g. an absorbent material). The absorbent body 751 surrounds a substantially rigid tip 755 (see FIG. 22). FIG. 22 shows a perspective view of the sample collection device 704 with the absorbent body 751 removed so as to reveal the substantially rigid tip 755. The sample collection device 704 further comprises a plunger 729. Similarly to previous embodiments, the device 704 comprises a main body 710 connected to the sample collection element 708 by a rod 782. As depicted, the substantially rigid tip 755 and the plunger 729 may be integrally formed with the rod 782, which may be integrally formed with the main body 710 of the device 704. The absorbent body 751 may be in the form of an absorbent foam, or any other absorbent body. The absorbent body 751 is also compressible. The absorbent body 751 may comprise a hollow core 753 in which the substantially rigid tip 755 is at least partially arranged. The plunger 729 may act to compress the absorbent body 751 and thereby expel liquid out of the absorbent body 751, as will be described in more detail below. FIG. 21 shows the sample collection device 704 with the absorbent body 751 in a first, non-compressed configuration. In this configuration, no part of the tip 755 protrudes out of the absorbent body 751, and thus a user may collect a sample, e.g. a urine sample, without risk of causing injury by the tip 755.

Use of the sample collection device 704 shown in FIGS. 21 and 22 will now be described with reference to FIGS. 23A-23C. FIGS. 23A-23C depict cross-sectional views through a a sample processing apparatus 702, viewed in the same direction as the cross-sections shown in FIGS. 20A-20C. using the sample collection device 704 shown in FIGS. 21 and 22 together with a sample processing device 706 which includes a housing 711 and tube 707 that are substantially the same as the housing and tube described above with reference to FIGS. 16-20C. The only notable difference is that the housing 711 does not include a projecting wall.

Once a user has collected a sample using the sample collection device 704, e.g. following the dipping of the sample collection element 708 into a liquid material, such that the absorbent body 751 has absorbed some liquid, a user may then insert the sample collection device 704, e.g. the sample collection element 708 thereof, into the cavity 732 of the sample processing device 706. At this point, the sample collection device 704 may not yet contact the first breakable seal 756. With reference to FIG. 23B, once the sample collection device 704 has been inserted by a sufficient amount, the first threaded portion 736 of the sample processing device 706 may engage with the second threaded portion 726 of the sample collection device 704. Advancement of the sample processing device 704 into the cavity 732 may then be achieved by relative rotation of the sample collection device 704 and the sample processing device 706. The sample collection device 704 may be rotated to a point at which the sample collection element 708, specifically the absorbent body 751, contacts the first breakable seal 756, as shown in FIG. 23B.

With reference to FIG. 23C, further advancement of the sample collection device 704, results in the plunger 729 pressing against the absorbent body 751 and compressing the absorbent body from the first, non-compressed configuration shown in FIG. 23B, to a second, compressed configuration, as shown in FIG. 23C. In this compressed configuration, the tip 756 protrudes at least as far forward as the absorbent body 751 and breaks, e.g. penetrates, the first breakable seal 713. The compression of the absorbent body 751, by the plunger 729, acts to force liquid out of the absorbent body, and into the first portion 747 of the tube 707. Before passing into the tube, the sample passes through the filter 713. The sample liquid may then be contained within tube 707, specifically the first portion 747 of the cavity 732, and mix with any reagent therein.

The plunger 729 may act to seal against the inside surface 731 of the cavity 732 and thus ensure that the sample is contained within the first portion 747 of the cavity 732. The first breakable seal 756 may be sufficiently strong that it does not break until contacted by the tip 755, i.e. it does not break under the compressive force applied by the absorbent body 751.

FIG. 24A shows a perspective view of a sample collection device 804 in accordance with another embodiment of the present invention. The sample collection device 804 comprises a sample collection element 808 in the form of a capillary tube configured to contain a liquid therein under the action of capillary forces. The sample collection device 804 further comprises a main body 810 to which a puncture element (not visible), arranged to move relative to a cover 861, is attached.

The sample collection device 804 comprises a seal breaking element 857 which may comprise at least one tooth 859 configured to break the first breakable seal 856 (see FIG. 24C). The sample collection element 808 may be arranged to move relative to the seal breaking element 857. As depicted in FIG. 24A, at least initially, the sample collection element 808 may protrude further at a distal end of the device 804 than the sample breaking element 857, such that a sample can be collected. This position of the sample collection element 808 may be considered to be a collection position. The sample collection device 804 comprises a hollow rod 882 (i.e. a tubular member) connected to the main body 810. An end of the hollow rod 882 may define the seal breaking element 857, as shown. The hollow rod 882 may be considered to form a sample collection element receiving portion as it receives at least part of the sample collection element 808. The sample collection element 808 may be arranged within the hollow rod 882 so as to be capable of moving relative to the hollow rod 882 between a collection position (as shown in FIG. 24A) and a retracted position, as shown in later Figures.

FIG. 24B shows a perspective view of the puncture element and associated cover 861. This part of the device may be considered to be a safety lancet. It may be an off-the-shelf component which is attached to the main body 810 of the sample collection device 804.

In use, a user may, for example, puncture their skin with the puncture element in order to obtain a blood sample. The retractable cover 861 may advantageously ensure that the puncture element is safely covered once it has been used. Once a user has punctured their skin, they may place the sample collection element 808, in the form of a capillary tube, into contact with the blood escaping via the puncture in the skin. Blood will then be drawn into the sample collection element 808 and be held therein under capillary forces.

FIG. 24C-24G show a cross-sectional view through a sampling apparatus 802 incorporating the sample collection device 804, viewed in the same direction as FIGS. 20A-20C. With reference to FIG. 24C, a user may insert the sample collection device 804 into a cavity 832 of a sample processing device 806. The sample processing device 806 comprises a housing 811 attached to a tube 807. The sample processing device 806 is identical to the sample processing device described above and shown in FIGS. 16-24C. As shown in FIG. 24D, a user may insert the sample collection device 804 such that the sample collection element 808 contacts the first breakable seal 856. The first breakable seal 856 and the sample collection element 808 may be configured such that the sample collection element 808, in the form of a capillary tube (as shown), is not able to break the first breakable seal.

With reference to FIG. 24E, as the sample collection device 804 is further advanced into the cavity 832, and once the first threaded portion 836 of the sample processing device 806 engages with the second threaded portion 826 of the sample collection device 804 (such that rotation of the sample collection device 804 relative to the sample processing device 806 drives linear movement of the sample collection device 804 into the cavity 832), the sample collection element 808 slides within the hollow core 877 of the hollow rod 882. The sample collection element 808 moves from the collection position, shown in FIGS. 24C and 24D, towards a retracted position. FIG. 24F shows the sample collection device 804 at the point at which it has been advanced relative to the sample processing device 806 to a point at which the sample collection element 808 has fully translated within the hollow core 877 of the hollow rod 882 to its retracted position. In this position, the seal breaking element 857 protrudes as far forward as the sample collection element 808.

Final advancement of the sample collection device 804, through continued rotation of the sample collection device 804 relative to the sample processing device 806 (through the engagement of the first and second threaded portions 836, 826) the sample collection device 804 is driven to the point at which the seal breaking element 857, e.g. the at least one tooth 859 thereon, breaks the first breakable seal 856. The seal breaking element 857 may eb configured, e.g. having a suitable arrangement of teeth or other protrusions, to break the first breakable seal 856, i.e. such that a sample can pass therethrough, but whilst leaving the first breakable seal 856 still attached within the device 806. This may advantageously prevent any parts of the first breakable seal 856 from physically mixing with sample (and indeed any reagent) which may otherwise contaminate, or complicate further analysis of, the sample.

Breaking of the first breakable seal 856 results in the sample 859 within the sample collection element 808 being exposed to the first portion 847 of the cavity 832, defined within the tube 807 of the sample processing device 806. Any reagent within the first portion 847 may then mix with the sample contained within the sample collection element 808.

FIG. 25 shows a perspective view of a sample collection device 904 which comprises a sample collection element in the form of a sample collection conduit 908. The sample collection conduit 904 comprises an inlet 907. The sample collection conduit 908 may sit within a sample collection device such that in at least one position, the inlet 907 extends to an outside of the device. As such a user may easily access the inlet 907, e.g. with their mouth, to provide a sample. The sample collection conduit 908 comprises a pointed tip 955 which may act as a seal breaking element. A plurality of apertures 963 are arranged at the pointed tip 955 and allow fluid to flow out of the sample collection conduit 908. The sample collection conduit 908 further comprises a plunger 929 which may seal against the inside surface of a cavity (of a sample processing device which the sample collection device 904 is used with) and act to force a liquid through the cavity. The sample collection device 904 further comprises a lid 910, which may have substantially the same shape as the main body of the sample collection device of earlier embodiments. The lid 910 may function to close off an open end of a sample processing device which the sample collection conduit 908 is used with, and may act to move the sample collection conduit 908 relative to the sample processing device.

FIG. 26 shows a perspective view of another embodiment of a sample collection device 1004. The sample collection device 1004 comprises a sample collection element in the form of a swab 1008. The device 1004 comprises a main body 1010 from which a hollow rod 1082 extends. The rod 1082 defines a seal breaking element 1057 which comprises a plurality of teeth 1159 configured to break the first breakable seal of a sample processing device. The hollow rod 1082 defines a hollow core 1077. The swab 1008 (i.e. the sample collection element) is configured to slide within the hollow core 1077 between a collection position (as shown in FIG. 26) and a retracted position (not shown). In this regard, the sample collection device 1004 may function in an identical manner to the sample collection device 804 described above.

FIG. 27 shows a perspective view of another embodiment of a sample collection device 1104. The sample collection device 1104 is identical to the sample collection device 1004 shown in FIG. 26, and described above, except for the sample collection element in the form of a swab 1008 instead comprises a curette 1108. The sample collection device 1104 may otherwise function in an identical manner.

FIG. 28 shows a perspective view of another embodiment of a sample collection device 1204. The sample collection device 1204 comprises a sample collection element 1208 which comprises a body 1265 which comprises a series of slots 1267 therein. The slots 1267 within the body 1265 increase the total surface area of the sample collection element 1208. As a result, the surface tension between a sample and the sample collection element 1208 may be increased. As a result, the sample collection element 1208 may be particularly well suited to collected samples with higher viscosities, e.g. mucus. Whilst slots 1267 are shown, it will be appreciated that the increase in surface tension may be achieved by any suitable means, e.g. through the presence of holes, protrusions etc. The sample collection device 1204 may otherwise function in an identical manner to the sample collection device 804 shown and described above, e.g. the sample collection element 1208 may be capable of moving between collection and retracted positions.

FIG. 29A shows a perspective view of another sample collection device 1304. The sample collection device comprises a sample collection element 1308 which comprises a sharpened portion 1369. The sharpened portion 1369 may be configured to penetrate a surface (e.g. skin). The sample collection element 1308 comprises a sample receiving portion 1373 which receives a sample. As with other embodiments, the sample collection element 1308 is able to move relative to a tubular body 1382 between the collection position, shown in FIG. 29A, into a retracted position, shown in later Figures. As with earlier embodiments, the sample collection device 1308 comprises a main body 1310. At one end of the main body 1310 an ejection button 1371 is arranged. The ejection button 1371 may be utilised to eject a sample out of the sample receiving portion 1373, as will be described in more detail with reference to later Figures below.

FIG. 29B shows a cross-sectional view through the sample collection device 1304 through the line A-A when viewed in the direction indicated by arrow B. This view shows the sample collection device 1304 when no sample has been collected therein. As depicted in FIG. 29C, which shows a corresponding view to that shown in FIG. 29B, when a user collects a sample, e.g. by piercing the surface of their skin using the sharpened portion 1369, a sample 1375, is collected with the sample receiving portion 1373.

FIGS. 29D-29G show cross-sectional views through a sample collection device 1304 and a sample processing device 1036 viewed in the same direction as shown in FIGS. 29B and 29C. With reference to FIG. 29D, the sample collection device 1304, e.g. the sample collection element 1308 thereof, is inserted into the cavity 1332 of the sample processing device 1306. At the position shown in FIG. 29D, the sample collection element has not yet contacted the first breakable seal 1356. With reference to FIG. 29E, the first threaded portion 1336 of the sample processing device 1306 and the second threaded portion 1326 of the sample collection device 1304 may engage with one another such that rotation of the sample collection device 1304 relative to the sample processing device 1306 drives the sample collection device 1304 further into the sample processing device 1306. As the sample collection device 1304 advances further, the sample collection element 1308 moves from the collection position towards a retracted position, by sliding within the hollow core 1377 of the hollow rod 1304.

With reference to FIG. 29F, once the sample collection device 1304 has been sufficiently advanced, the seal breaking element 1357 is at least as far forward as the sample collection element 1308 and further movement causes the seal breaking element 1357 to break the first breakable seal 1356.

Once the first breakable seal 1356 has been broken, it may then be desirable to eject the sample 1375 from the sample collection element 1308. With reference to FIG. 29G, a user may press on the ejection button 1371 which may push an ejection element 1379 (e.g. in the form of a rod) into the sample receiving portion 1373 thereby ejecting the sample 1375. The sample 1375 may then be suitably processed within the first portion 1347 within the tube 1307. Ejecting the sample 1375 in a controlled manner within the sample processing device 1306 may ensure that a sample can be safely collected, ejected and processed, with minimal human/environmental interaction, thereby potentially minimising the risk of contamination of the sample.

FIG. 30 shows a perspective view of another embodiment of a sample collection device 1404. The sample collection device 1404 comprises a sample collection element comprising a gripping arrangement 1408. The gripping arrangement 1408 comprises a first member 1481 and a second member 1483. The first member 1481 is arranged so as to move relative to the second member 1483. The first member 1481 is connected to the second member 1483 via a living hinge 1494. The living hinge 1494 may act to resiliently bias the first member 1481 into the position shown in FIG. 30, which may be considered to be an open position. A rod 1487, not visible in FIG. 30, extends through a hollow core 1477 of a hollow rod 1482. The hollow rod 1482 extends from a main boy 1410 of the device 1404. The rod 1487 terminates in a grip member 1485 which is arranged at a proximal end of the device 1404. The hollow rod 1482 may also define a sample breaking element 1457, which may comprise a plurality of teeth 1459, in a similar manner to earlier embodiments.

In the embodiment shown in FIG. 30, the hollow rod 1482 also functions as a driving arrangement, specifically forming a driving structure, which acts to move the first member 1481 into the closed position, as will be described in more detail further below.

FIG. 31 shows a perspective view of the gripping arrangement 1408, comprising the first and second member 1481, 1483, the connecting rod 1487 and the gripping element 1485. As shown in this Figure, the first member 1481 may comprise a specific profiled portion 1489 which may act to guide movement of the first member 1481. FIG. 32 shows a perspective view of the main boy 1410 and the hollow rod 1482 in isolation, more clearly showing the hollow core 1477.

Whilst in this embodiment, first and second members 1481, 1483 are shown and described, it will be appreciated that the gripping arrangement 1408 may comprise any suitable number of members. Further, whilst it is shown and described how the first member 1481 moves relative to the second member 1483, the second member 1483 may also move relative to the first member 1481.

FIG. 33A-33G show cross-sectional views through the line A-A of the sample collection device in the direction indicated by the arrow B, as shown in FIG. 30. FIG. 33A shows the point at which the sample collection device 1404 has been moved into a position in which a sample 1475 is positioned within the gripping arrangement 1408 (i.e. between the first and second members 1481, 1483). Once suitably positioned, a user may then proceed to move the hollow tube 1482 (i.e. the driving arrangement and driving structure) relative to the gripping arrangement 1408. This is depicted in FIG. 33B. This movement may be achieved by a user gripping the main body 1410 in one hand, and gripping the gripping member 1485, in another, and moving the main body 1410 away from the gripping member 1485. As a result, this creates a gap X between the gripping member 1485 and main body 1410. As the forward end 1457 (which also function as the seal breaking element) of the hollow tube 1482 contacts the first member 1481 it is caused to pivot towards the second member 1482.

With reference to FIGS. 33C-33E, this movement is continued, bringing the first member 1481 gradually closer to the second member 1482, until the sample 1475 is gripped between the first and second member 1481, 1483, as shown in FIG. 33E. At this point, a user may continue to separate the main body 1410 from the gripping member 1485, resulting in the gripping arrangement 1408 being pulled into the hollow core 1477, as shown in FIGS. 33F and 33G. Once sufficiently retracted, the gripping arrangement 1408 may be positioned such that it only protrudes as far, or even not as far, forward as the forward end 1457 of the hollow tube 1482 (see FIG. 33G). As a result, the sample 1375 may be safely contained within the hollow tube 1482.

FIG. 33H shows a cross-sectional view of the sample collection device 1404 being inserted into a sample processing device to the point at which the sample collection device 1404, e.g. the seal breaking element 1457, has broken the first breakable seal 1456. The sample collection device 1404 may be advanced relative to the sample processing device 1406 in a manner similar to the embodiments described above.

Once the sample collection device 1404 has broken the first breakable seal, it may then be desirable to release the sample 1408 from the gripping arrangement 1408. Accordingly, with reference to FIG. 33I, the gripping member 1485 may be pushed, relative to the main body 1410, thereby pushing the gripping arrangement 1408 out of the hollow tube 1482. The gripping element 1485 may be pushed until the point at which the gripping arrangement 1408 is sufficiently far enough out of the hollow tube 1482 such that the first member 1481 is able to move away from the second member 1482, e.g. under a resilient bias provided by the living hinge 1494. Movement of the first member 1481 in this manner will release the sample 1475 such that it is free to move within the first portion 1447 of the cavity 1432 of the sample processing device 1406, specifically within the tube 1407, as shown in FIG. 33J. The Applicant has appreciated that this arrangement may allow a user to controllably grip and release a sample within a sample processing device.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

1. A sampling apparatus comprising:
a sample collection device comprising a sample collection element for the collection of a sample; and
a sample processing device comprising a cavity shaped to receive at least a portion of the sample collection device, wherein:
the cavity comprises a first portion closed off by a first breakable seal; and
wherein the cavity is shaped such that when the sample collection device is inserted into a first position in the cavity, the sample collection device breaks the first breakable seal so as to allow a sample collected by the sample collection element to pass into the first portion of the cavity.

2. The sampling apparatus of claim 1, wherein the sample collection element is configured to break the first breakable seal.

3. The sampling apparatus of any preceding claim, wherein the sample processing device comprises a housing, and wherein the cavity is at least partially formed within the housing.

4. The sampling apparatus of claim 3, wherein the cavity comprises a proximal end into which the sample collection element is inserted, and a distal end, wherein the distal end is closed by an integrally formed part of the housing.

5. The sampling apparatus of claim 3, wherein the cavity comprises a proximal end into which the sample collection element is inserted, and a distal end, wherein the distal end is closed by a closure member attached to the housing.

6. The sampling apparatus of claim 5, wherein the closure member comprises a collection tube.

7. The sampling apparatus of any preceding claim, wherein the first breakable seal contains a reagent within the first portion of the cavity.

8. The sampling apparatus of any preceding claim, wherein the sample collection device is configured to seal the cavity at least when the sample collection device is in the first position.

9. The sampling apparatus of any preceding claim, wherein the sample processing device further comprises a second breakable seal, spaced from the first breakable seal and arranged to define a chamber within the cavity.

10. The sampling apparatus of claim 9, wherein the sample collection device is configured to break the second breakable seal when the sample collection device is in a second position within the cavity.

11. The sampling apparatus of any preceding claim, wherein the sample processing device comprises a processing chamber arranged fluidly downstream of the first breakable seal.

12. The sampling apparatus of any preceding claim, the sample processing device further comprises a sample analysis means.

13. The sampling apparatus of any preceding claim, wherein the sample processing device comprises a first threaded portion and the sample collection device comprises a second, corresponding threaded portion, each having a first handedness and arranged such that the second threaded portion engages the first threaded portion when the sample collection device is inserted into the cavity and such that rotation of the sample collection device relative to the sample processing device in a first rotational direction advances the sample collection device into the cavity in a first direction.

14. The sampling apparatus of claim 13, wherein the first threaded portion and second corresponding threaded portion are arranged such that rotation of the sample collection device during connection of first threaded portion and second corresponding threaded portion advances the sample collection device into the first position whereby the first breakable seal is broken.

15. The sampling apparatus of claim 13 or 14, wherein the sample processing device comprises a third threaded portion, and the sample collection device comprises a fourth, corresponding threaded portion, each having a second handedness, different to the first handedness, and arranged such that the fourth threaded portion engages the third threaded portion when the sample collection device is within the cavity and such that rotation of the sample collection device relative to the sample processing device in a second, different, rotational direction advances the sample collection device into the cavity in the first direction.

16. The sampling apparatus of claim 15, wherein the third threaded portion and fourth corresponding threaded portion are arranged such that rotation of the sample collection device during connection of third threaded portion and fourth corresponding threaded portion advances the sample collection device into a second position whereby a second breakable seal is broken.

17. The sampling apparatus of any of claims 15 or 16, wherein the third and fourth threaded portions are arranged such that they engage one another following engagement of the first and second threaded portions.

18. The sampling apparatus of any preceding claim, wherein at least one of the sample collection device or the sample processing device comprises a movement retraction means which prevents the sample collection device from being separated from the sample processing device once it has been inserted into the cavity by a predetermined distance.

19. The sampling apparatus of any preceding claim, wherein the sample collection element comprises at least one of: a sample collection tube, a capillary tube, a saliva collection tube, a sample collection patch, a sample collection swab, a curette, a syringe and a pipette.

20. The sampling apparatus of any preceding claim, wherein the sample collection device is configured to couple to the sample processing device in a manner in which at least in one position of the sample collection device relative to the sample processing device, the sample collection device is rotationally coupled to the sample processing device such that rotation of the sample collection device drives rotation of the sample processing device.

21. The sampling apparatus of any preceding claim, wherein the sample processing device comprises a filter arranged within the cavity to filter the sample.

22. The sampling apparatus of any preceding claim, wherein the sample collection device comprises a main body and wherein the main body comprises a sample collection element receiving portion shaped so as to receive one of a plurality of different sample collection elements.

23. A sample collection device, for collecting a sample, comprising:
a main body, wherein the main body comprises a sample collection element receiving portion shaped so as to receive one of a plurality of different sample collection elements.

24. The sampling apparatus or sample collection device of any one of claims 22 or 23, wherein the plurality of different sample collection elements comprise at least two of: a sample collection tube, a capillary tube, a saliva collection tube, a sample collection patch, a sample collection swab, a curette, a syringe, a gripping arrangement, an absorbent body and a pipette.

25. The sampling apparatus or sample collection device of any one of claims 22 to 24, wherein the sample collection element receiving portion is dimensioned to receive the sample collection element in a first position therein, such that the sample collection element protrudes by a first distance from an end of the sample collection element receiving portion, and further a second position wherein the sample collection element protrudes by a second, reduced distance, from the end of the sample collection element receiving portion.

26. The sampling apparatus or sample collection device of claim 25, wherein the sample collection element receiving portion is configured such that irrespective of the sample collection element which is received therein, when in the second position, the sample collection elements protrudes from the end of the sample collection element receiving portion by the second distance.

27. The sampling apparatus or sample collection device of any preceding claim, wherein the sample collection element comprises a substantially rigid tip surrounded by an absorbent body, wherein the absorbent body is compressible such that in a first, non-compressed configuration, the tip does not protrude further than an end of the absorbent body and in a second, compressed configuration, the tip protrudes at least as far as an end of the absorbent body.

28. The sampling apparatus or sample collection device of any preceding claim, wherein the sample collection device comprises a seal breaking element.

29. The sampling apparatus or sample collection device of claim 28, wherein the seal breaking element is configured to break the first breakable seal in a manner in which at least part of the seal remains attached to a wall of the cavity.

30. The sampling apparatus or sample collection device of claim 28 or 29, wherein the sample collection element is arranged to move relative to the seal breaking element between a collection position in which the sample collection element protrudes further than the seal breaking element, at a distal end of the sample collection device, and a retracted position, in which the seal breaking element protrudes at least as far the sample collection element, at the distal end of the sample collection device.

31. The sampling apparatus or sample collection device of any preceding claim, wherein the sample collection element may comprise a body which comprises a plurality of at least one of: grooves, slots, holes, protrusions formed therein, configured to increase the surface tension between the body and the sample, and thereby hold the sample against the body.

32. The sampling apparatus or sample collection device of any preceding claim, wherein the sample collection element comprises a sharpened portion configured to penetrate a surface and a sample receiving portion into which a sample is received.

33. The sampling apparatus or sample collection device of any preceding claim, wherein the sample collection element comprises a gripping arrangement comprising first member and second member, wherein at least the first member is configured to move between an open position, in which the first member is spaced from the second member so as to receive, in use, a sample therebetween, and a closed position in which the first member is moved towards the second member so as to grip the sample between the first and second members.

34. The sampling apparatus or sample collection device of claim 33, wherein the sample collection device comprises a driving arrangement configured to drive the first member from the open position to the closed position.

35. The sampling apparatus or sample collection device of claim 34, wherein the driving arrangement comprises a driving structure arranged to drive the first member into the second position, and wherein driving structure is movably mounted so as to be capable of moving relative to the gripping arrangement, and arranged such that when the driving structure is moved in a first direction the first member is driven from the open position to the closed position.

36. The sampling apparatus or sample collection device of any of claims 33 to 35, wherein the first member is resiliently biased into the open position.

37. The sampling apparatus or sample collection device of claim 35 or 36, wherein the driving structure is configured to move relative to the gripping arrangement into a position whereby a forward end of the driving structure is at least as far forward as the gripping arrangement, and wherein the forward end defines a seal breaking element.

38. The sampling apparatus or sample collection device of any of claims 33 to 37, wherein the first member is mounted to move relative to the second member by a living hinge.

39. The sampling apparatus or sample collection device of any preceding claim, wherein the sample collection element comprises a tip and a surrounding wall which extends around the tip and which defines a sample collection volume between the wall and the tip.

40. The sampling apparatus of claim 39, wherein the sample processing device comprises a projecting wall which extends away from the first breakable seal, and the projecting wall and the surrounding wall may be dimensioned such that as the sample collection device is inserted into the cavity of the sample processing device, the tip passes through an inner side of the projecting wall and the surrounding wall passes around an outer side of the projecting wall.

41. A kit of parts comprising:
a sample collection device of any of claims 23 to 26 configured to be capable of receiving a plurality of different sample collection elements; and
a plurality of different sample collection elements.

42. The kit of parts of claim 41, further comprising a sample processing device.

43. The kit of parts of claim 41 or 42, further comprising a plurality of capsules each comprising different reagents and/or different volumes of reagents.

44. A medical device comprising:
a first body and a second body, wherein the first body is arranged to move relative to the second body;
a first thread arrangement configured between the first body and the second body such that rotation of the first body in a first rotational direction relative to the second body advances the first body in a first linear direction; and
a second thread arrangement configured between the first body and the second body such that rotation of the first body in a second direction opposite to the first rotational direction advances the first body in the same first linear direction.

45. The medical device of claim 44, wherein:
the first thread arrangement comprises a first threaded portion arranged on the first body and a second corresponding threaded portion on the second body;
the second thread arrangement comprises a third threaded portion arranged on the first body and a fourth threaded portion arranged on the second body; and wherein
the first and second threaded portions have an opposite handedness to the third and fourth threaded portions.

46. The medical device of claim 45, wherein the first and third threaded portions are displaced along on the first body in a direction aligned with the first linear direction and/or wherein the second and fourth threaded portions are displaced along the second body in a direction aligned with the first linear direction.
